(19) United States
(12) Patent Application Publication
(10) Pub. No.: US 2025/0082663 A1
(43) Pub. Date: Mar. 13, 2025

(54) COMPOUNDS AND METHODS FOR TREATING OR PREVENTING AUTOSOMAL DOMINANT POLYCYSTIC KIDNEY DISEASE (ADPKD), AUTOSOMAL DOMINANT POLYCYSTIC LIVER DISEASE (ADPLD) AND/OR AUTOSOMAL RECESSIVE POLYCYSTIC KIDNEY DISEASE (ARPKD)

(71) Applicant: YALE UNIVERSITY, New Haven, CT (US)

(72) Inventors: Matteus KRAPPITZ, New Haven, CT (US); Sorin FEDELES, New Haven, CT (US); Stefan SOMLO, New Haven, CT (US)

(21) Appl. No.: 18/569,379

(22) PCT Filed: Jun. 14, 2022
(86) PCT No.: PCT/US2022/072926

§ 371 Date: Dec. 12, 2023

Related U.S. Application Data
(60) Provisional application No. 63/210,726, filed on Jun. 15, 2021.

(51) Int. Cl. A61K 31/7064

(52) U.S. Cl. CPC A61K 31/7064

(57) ABSTRACT

The present invention provides methods for treating or preventing Autosomal Dominant Polycystic Kidney Disease (ADPKD) and/or Autosomal Dominant Polycystic Liver Disease (ADPLD) and/or Autosomal Recessive Polycystic Kidney Disease (ARPKD) in a mammal.

### CROSS-REFERENCE TO RELATED APPLICATIONS

The present application claims priority under 35 U.S.C. § 119(e) to U.S. Provisional Patent Application No. 63/210,726, filed Jun. 15, 2021, which is incorporated herein by reference in its entirety.

### BACKGROUND

Autosomal dominant polycystic kidney disease (ADPKD) is the most common hereditary renal disease, occurring in 1:400 to 1:1,000 individuals. It accounts for over 90% of all hereditary renal cystic diseases and is characterized by the presence of bilateral renal cysts, which typically grow and expand slowly over decades resulting in significantly increased total kidney volume, progressive renal injury, and ultimately end stage renal disease (ESRD) around the sixth decade of life. The most common extra-renal manifestation of ADPKD is polycystic liver disease which can also occur as an independent genetic entity, Autosomal Dominant Polycystic Liver Disease (ADPLD). Further, the recessive form of PKD, Autosomal Recessive Polycystic Kidney Disease (ARPKD), is a rare inherited form of PKD caused by mutations in PKHD1, which encodes fibrocystin/polyductin (FPC).

There is thus a need in the art for identifying compounds and compositions that can be used to treat and/or prevent ADPKD and/or ADPLD and/or ARPKD. The present disclosure addresses this need.

### SUMMARY

In certain aspects, the present invention is directed to the following non-limiting embodiments.

In some embodiments, the present invention is directed to a method of treating or preventing Autosomal Dominant Polycystic Kidney Disease (ADPKD) and/or Autosomal Dominant Polycystic Liver Disease (ADPLD) and/or Autosomal Recessive Polycystic Kidney Disease (ARPKD) in a mammal.

In some embodiments, the method includes administering to the mammal a therapeutically effective amount of an IRE1α endonuclease activity inhibitor.

In some embodiments, the inhibitor does not inhibit IRE1α kinase activity.

In some embodiments, the inhibitor includes a peptide, antibody, small molecule, antagonist, aptamer, or peptidomimetic, and any combinations thereof.

In some embodiments, the small molecule comprises toyocamycin, or a salt, solvate, tautomer, geometric isomer, enantiomer, and/or diastereoisomer thereof.

In some embodiments, the administration has at least one of the following effects; (a) slows or inhibits development of at least one cyst in the mammal's kidney or liver; (b) kills or prevent growth or multiplication of at least one cyst cell; (c) selectively kills or prevents growth or multiplication of at least one cyst cell as compared to at least one non-cyst cell.

In some embodiments, the inhibitor is the only therapeutically effective agent administered to the mammal.

In some embodiments, the inhibitor is the only therapeutically effective agent administered to the mammal in an amount sufficient to treat or prevent ADPKD and/or ADPLD and/or ARPKD in the mammal.

In some embodiments, the mammal is further administered at least one additional agent that treats or prevents ADPKD and/or ADPLD and/or ARPKD.

In some embodiments, the at least one additional agent is Tolvaptan, or a salt, solvate, tautomer, geometric isomer, enantiomer, and/or diastereoisomer thereof.

In some embodiments, the mammal is human.

In some embodiments, the inhibitor is administered to the mammal by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous routes.

### BRIEF DESCRIPTION OF THE DRAWINGS

The following detailed description of illustrative embodiments of the invention will be better understood when read in conjunction with the appended drawings. For the purpose of illustrating the invention, certain illustrative embodiments are shown in the drawings. It should be understood, however, that the invention is not limited to the precise arrangements and instrumentalities of the embodiments shown in the drawings.

FIG. 1 illustrates a diagram view of the unfolded protein response with the three effector pathways: IRE1α, PERK, and ATF6 (adapted from slideplayer dot com/slide/7924168).

FIG. 2 illustrates a schematic depiction of IRE1α dependent pathways, i.e. acting either via XBP1 in order to increase adaptation and recovery under stress or IRE1α-driven XBP1-independent to trigger a pro-apoptotic response under circumstances of unresolved chronic stress (Gorman et al., 2012, Pharmacology and Therapeutics 134(3):306-316).

FIGS. 3A-3B illustrate the finding that Pkd1 and Xbp1 interact at the genetic level. FIG. 3A: Inactivation of XBP1 can slow down progression of polycystic kidney disease in an early ADPKD model resulting from collecting duct specific inactivation of polycystin-1 (middle panel). This effect is specific to XBP1 as restoration of its expression of active XBP1s in the Pkd1-XBP1 double knockout animals via a ROSA-flox-stop transgene (right panel) leads to similar progression of disease as seen in the Pkd1-only knockout mice (left panel). FIG. 3B: Morphological (KW/BW ratio, cystic index) and functional parameters (serum creatinine) are significantly improved on the Pkd1-XBP1 double knockout (DKO) background vs. Pkd1 single knockout (SKO) background and the DKO+XBP1s transgene re-expression (DKO+ABP1-TG) background. n=6,5,4.

FIGS. 4A-4F illustrate the finding that inactivation of XBP1 induces apoptosis specifically in kidney segments where Pkd1 was inactivated (Pkd1 cystic epithelia). FIG. 4A: The presence of apoptosis was examined by TUNEL staining on cryosections from Pkd1^{fl/fl};Pkhd-Cre (SKO), Pkd1^{fl/fl};Xbp1^{fl/fl};Pkhd1-Cre (DKO), or DKO mice expressing a ROSA-floxstop-XBP1 transgene (DKO-TG). FIG. 4B: Quantification of the apoptotic rates examined in panel A. FIG. 4C: PARP cleavage is increased in the DKO animals compared to either SKO or DKO+XBP1-TG. FIGS. 4D-4E: Proliferation rates as assessed by Ki67 staining were not different between SKO vs. DKO animals.

FIGS. 5A-5C illustrate the finding that Xbp1 inactivation leads to apoptosis of Pkd1 null cells in an adult whole nephron knockout ADPKD model. FIG. 5A: Inactivation of XBP1 in an adult ADPKD model (Pkd1^{fl/fl}; Pax8^{rtTA}; tet-OCre) leads to a dramatic improvement in the cystic phenotype compared to the Pkd1-only knockout. FIG. 5B: This effect is recapitulated by an improvement in kidney to body weight ratio and serum BUN. FIG. 5C: The presence of apoptosis was observed by TUNEL staining on cryosections from Pkd1^{fl/fl};XBP1^{fl/fl};Pax8^{rtTA};tet-OCre animals. No apoptosis was seen in the Pkd1^{fl/fll};Pax8^{rtTA};tet-OCre animals (data not shown). n=3,6.

FIG. 6 illustrates the finding that toyocamycin treatment in Pkd1^{RW/fl}; Pkhd1-Cre (neonatal ADPKD model) mice leads to a dramatic improvement in the cystic phenotype as seen by morphological (top panel) and functional (bottom panel) parameters.

FIG. 7 illustrates the finding that toyocamycin treatment in Pkd1^{RW/fl}; Pax8^{rtTA};tet-OCre mice (adult model of ADPKD) leads to a dramatic improvement in the cystic phenotype as seen by morphological (top panel) and functional (bottom panel) parameters. Pkd1 gene knockout was induced between 4 to 6 weeks age, and toyocamycin treatment was begun at 6 weeks and continued until 18 weeks age when the kidneys were examined.

FIG. 8 illustrates the Irela-dependent cells death pathway, as well as the finding the Irela and downstream pathways are activated in the Pkd1/Xbp1 DKO mice. This activation is reversed by re-expression of the active XBPIs from the conditional transgene.

FIG. 9 illustrates the finding that Ire1α inactivation does not impact ADPKD progression in absence of Pkd1.

FIG. 10 illustrates the finding that apoptosis is not impacted in the Pkd1-Ire1α DKO mice. Together with the data in FIG. 9, this demonstrates that the Ire1α kinase-dependent pro-apoptosis function drives the beneficial effect on the Pkd1-Xbp1 DKO background. In certain non-limiting embodiments, inhibitors of Ire1α endoribonuclease function, which are not Ire1α kinase inhibitors, have beneficial effects in ADPKD models.

FIG. 11: Mechanism of cyst formation.

FIG. 12: Approach to the treatment of ADPKD.

FIG. 13: Schematic of the IRE1-Xbp1 pathway. Ire1α activates XBP1 as part of the unfolded protein response (UPR). Research has shown that: XBP1 is not required for kidney homeostasis, XBP1 is not upregulated in ADPKD models, and XBP1 displays a genetic interaction with ADPKD genes.

FIGS. 14A-14E: XBP1 protects cystic cells from apoptosis in a neonatal ADPKDmodel. Neonatal gene inactivation model. Constitutive collecting duct specific Cre turns on at P1; phenotype assessed at P24.

FIGS. 15A-15B: The rescue effect due to XBP1 deletion is cell autonomous.

FIGS. 16A-16C: XBP1 deletion slows disease progression in an adult ADPKD model. Adult onset model (Pkd1fl/fl;Pax8rtTA;tet-OCre) based on doxycycline-induced gene deletion throughout the nephron. Inactivation between P28 and P42; phenotype assessed at 18 wks. FIGS. 17A-17D: The presence/activation of Ire1α in the absence of XBP1 drives apoptosis and disease improvement.

FIGS. 18A-18B: Ire1α inhibitor (toyocamycin) inhibits cystic cell viability.

FIGS. 19A-19B: Ire1α inhibitor improves disease progression in an adult ADPKD model. Adult onset model with complete loss of function (Pkd1fl/fl;Pax8rtTA;tet-OCre). Treatment started immediately after gene inactivation (from 6 to 18 weeks).

FIGS. 20A-20B: Ire1α inhibitor prevents cyst growth in the Pkd1^{RW/flox} adult model. Adult onset with a PC1 missense mutation in trans with loss of function. Gene inactivation between P28 and P42. Treatment (0.5 mg/kg; 1×/2 wks) between 6-18 weeks of age.

FIGS. 21A-21B: Adult onset with a PC1 missense mutation in trans with loss of function. Gene inactivation between P28 and P42. Treatment (0.5 mg/kg; 1×/2 wks) between 6-18 weeks of age. Inhibitor: 0.5 mg/kg IP.

FIGS. 22A-22C: Irela inhibitor prevents cyst growth in preclinical models. No apparent systemic toxicity (body weight). Reduced cyst growth (kidney/body weight ratio). Normalized kidney function (blood urea nitrogen [BUN]). Enhanced apoptosis specifically in cyst cells with PC1 mutation.

FIGS. 23A-23C: Ire1α inhibitor causes apoptosis of cyst lining epithelia. Adult onset with a PC1 missense mutation in trans with loss of function. Gene inactivation between P28 and P42. Treatment (0.5 mg/kg; 1×/2 wks) between 6-18 weeks of age.

FIGS. 24A-24B: Ire1α inhibitor prevents collagen deposition in preclinical models.

FIGS. 25A-25D: Therapeutic trial of Ire1α inhibitor in Pkd1^{R2216W/fl} mice. Gene inactivation between P28 and P42. Treatment (0.5 mg/kg; 1×/2 wks) between 13 (when disease is of moderate severity; see inset) to 18 wks (3 injections).

FIG. 26: Treatment with the Ire1α inhibitor does not cause liver toxicity. Adults onset model (complete loss of PC1); disease induced at P28 and treatment started at 13 wks until 18 wks; Inhibitor regimen: 0.5 mg/kg IP, 1×/week.

FIG. 27A: Inactivation of XBP1 can slow down progression of polycystic kidney disease due to inactivation of polycystin-1.

FIG. 27B: KW/BW ratio, cystic index and serum creatinine levels are significantly decreased in the Pkd1/XBP1 DKO animals compared with Pkd1 single knockout mice.

FIGS. 28A-28C: Inactivation of XBP1 induces apoptosis specifically in kidney segments where Pkd1 was inactivated. FIG. 28A: The presence of apoptosis was examined by TUNEL staining on cryosections from Pkd1^{fl/fl};Pkhd1-Cre (SKO), Pkd1^{fl/fl};Xbp1^{fl/fl};Pkhd1-Cre (DKO), or DKO mice expressing a ROSA-floxstop-XBP1 transgene (DKO-TG). FIG. 28B: Quantification of the apoptotic rates examined in FIG. 28A. FIG. 28C: PARP cleavage is increased in the DKO animals compared to either SKO or DKO+XBP1-TG.

FIG. 29A: Inactivation of XBP1 in an adult ADPKD model can slow down progression of polycystic kidney disease.

FIG. 29B: Quantitation of the cystic phenotype based shows significant improvement in KW/BW ratio (***p<0.001) and BUN (***p<0.001).

FIG. 29C: Apoptosis in increased in the Pkd1/XBP1 DKO kidneys as seen via TUNEL staining (red).

FIG. 30A: Deletion of Ire1α does not impact progression of polycystic kidney disease due to inactivation of polycystin-1 (right panel).

FIG. 30B: Morphological (KW/BW ratio and cystic index) and functional (BUN) parameters were not different between Pkd1 single and Pkd1/Ire1α double knockout kidneys.

FIG. 30C: Apoptosis was not different between Pkd1 single and Pkd1/Ire1α double knockout animals.

FIGS. 31A-31B: Toyocamycin treatment (0.5 mg/kg every 2 weeks) between 12 and 18 results in a significant improvement in the cystic phenotype as seen via a decrease in KW/BW ratio (*p=0.03). Inset, structure of toyocamycin.

FIGS. 32A-32B: Toyocamycin treatment (0.5 mg/kg at P10) of a neonatal model leads to significantly improved disease at P16.

FIGS. 33A-33B: Treatment of adult mice with toyocamycin (0.5 mg/kg; 1×/2 weeks) leads to a dramatic improvement in the cystic phenotype as seen via KW/BW ratio and BUN. Shown in the inset is a qPCR plot of spliced XBP1 levels between untreated (black bar) and treated (grey bar) mice showing decreased expression in the latter.

FIG. 34: Treatment of Pkd1^{R2216W/flox}; Pax8rtTA; tet-OCre adult mice with toyocamycin (0.5 mg/kg; 1×/2 weeks) leads to apoptosis of cyst lining epithelia. TUNEL (red) delineates cells undergoing programmed cell death. LTA (green) marks proximal tubules.

FIG. 35: Dose response curves of mouse-derived (Pkd1^{+/-}, Pkd1^{-/-}, and Pkd1^{-/-};XBP1^{-/-} mouse kidney cells treated with an ROS stress inducer (11β-dichloro) demonstrate that XBP1 deletion sensitized Pkd1 null cells towards apoptosis. The viability response was measured by a metabolic fluorescent dye (Cell-titer Blue).

FIG. 36: Gene inactivation between P28 and P42. Treatment (0.5 mg/kg; 1×/2 wks) between 6-18 weeks of age.

### DETAILED DESCRIPTION

The present invention is based in part on the discovery of a novel genetic and functional interaction between Pkd1 and XBP1, which is critical for controlling the viability of mutant cells that transform and proliferate to form kidney and liver cysts in the setting of ADPKD. The IRE1α-XBP1 pathway is mainly dormant in the development of the kidney, and has been extensively examined in the context of oncogenesis, and thus also represents an attractive target from a drug-development perspective. While most therapeutic strategies to date in ADPKD have focused on anti-proliferative or antisecretory targets (i.e. the only FDA approved therapy: Tolvaptan, also known as N-(4-{[(5R)-7-Chloro-5-hydroxy-2,3,4,5-tetrahydro-1H-1-benzazepin-1-yl]carbonyl}-3-methylphenyl)-2-methylbenzamide), the present approach via chemical inhibition of the IRE1α-XBP1 pathway aims to specifically ablate mutant cyst-forming cells and slow down cyst growth by enhancing selective cell death. In certain embodiments, as a stand-alone therapy, the mode of application is a pulse therapy with long intervals rather than the continuous application of Tolvaptan (applied twice daily). In other embodiments, this mode is beneficial from a side-effect and compliance perspective. In yet other embodiments, the pulse therapy with toyocamycin is complementary to long-term therapies that target proliferation, and can lead to “synthetic lethality” of the disease-driving cystic cells. This can allow for reduced dosing requirements for any single drug, and thereby keep the drugs well within their therapeutic indices.

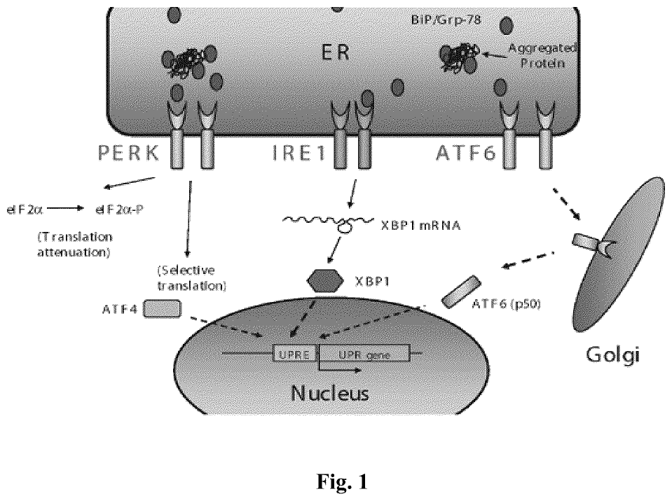

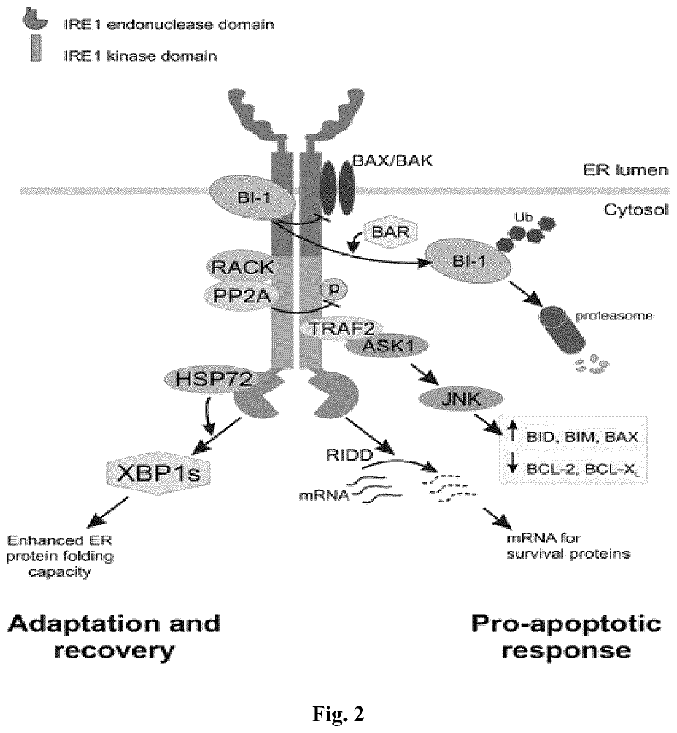

ADPLD and ADPKD, despite differential kidney manifestations, share a common underlying molecular genetic mechanism centered on the activity of polycystin-1 (PC1), the protein product of the major gene for ADPKD. The IRE1α-XBP1 pathway, the most conserved branch of the ER unfolded protein response (UPR) (FIGS. 1-2), plays a protective role in cyst formation induced by Sec63p deficiency by modulating the folding environment of misfolded polycystin-1 via up-regulation of a chaperone program dependent on XBP1.

Activated IRE1α splices X-box-binding protein 1 (XBP1) mRNA via IRE1α endoribonuclease function to generate a potent transcriptional activator, XBP1s. While there is no overt activation of UPR as indicated by absence of increased splicing of XBP1s in ADPKD cysts in the kidney, the present studies found evidence of a critical role for the basal homeostatic level of XBP1s in ADPKD cysts due to inactivating mutations in polycystin-1.

XBP1 is a genetic interactor of Pkd1 and can promote the progression of ADPKD resulting from inactivation of PC1 by protecting Pkd1 cyst cells from apoptosis. Double inactivation of Pkd1 and XBP1 leads to specific apoptosis of cyst lining epithelia without any impact on proliferation and no discernible effects on cells that still express one normal copy of Pkd1.

The basal activity of spliced active XBP1s appears to be a protective survival factor for PC1-null cells that form cysts. In certain embodiments, the data indicates that modulation of homeostatic IRE1α-XBP1 signaling in vivo is therapeutically beneficial, by selectively promoting the apoptosis of cells that have acquired second hits in Pkd1, and which are responsible for the initiation of cystic lesions that eventually lead to polycystic kidney and liver disease. Thus, specific pharmacological inhibition of the endoribonuclease activity of IRE1α, by inhibiting its ability to splice XBP1 to active XBP1s, should be beneficial in slowing cyst growth in ADPKD. This hypothesis was tested pharmacologically by inhibiting IRE1α endoribonuclease activity using toyocamycin, which is a potent IRE1α endoribonuclease inhibitor. Indeed, treatment of PKD mice with the drug lead to a dramatic reduction in kidney to body weight ratio and improved kidney function as seen by a reduction in serum creatinine and BUN. Targeting the cells that drive the progression of the disease by enhanced apoptosis due to inhibition of XBP1s slows the development of cysts and ameliorates polycystic kidney disease in both early onset and adult mouse models based on the Pkd1 orthologous gene.

### Description

Based on the common involvement of PC1 and Ire1α-XBP1 in proliferative neoplastic or malignant backgrounds, it was investigated whether XBP1 can modulate ADPKD progression via a direct genetic interaction with Pkd1.

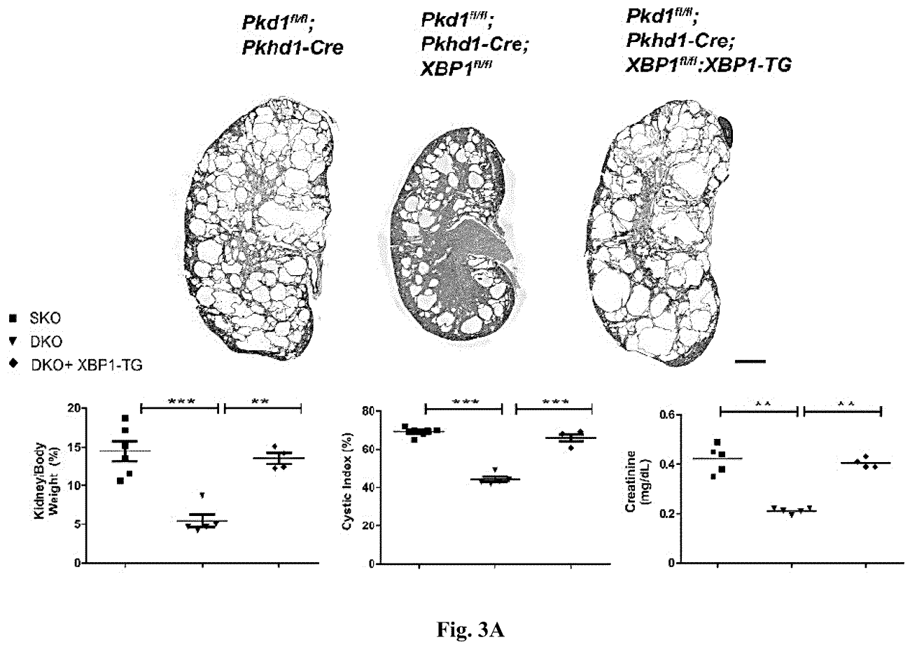

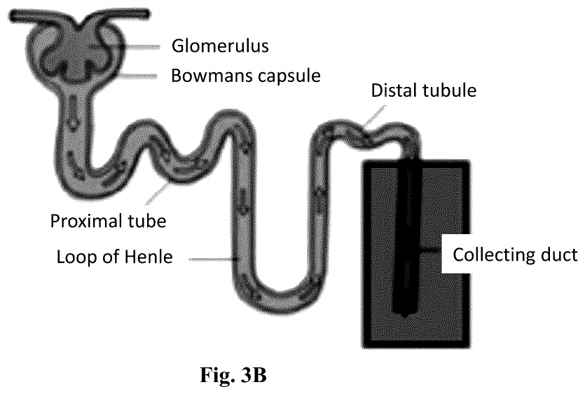

Relevant murine models of kidney Pkd1 and XBP1 inactivation were used to test this interaction. An established early inactivation model for ADPKD is the Pkhd1-Cre; Pkd1^{fl/fl} in which Pkd1 is selectively and completely inactivated in the collecting duct by postnatal day seven (P7) (Ma, et al., 2013, Nat Genet 45:1004-1012). To this end, Pkd1^{fl/fl}; Pkhd1-Cre (SKO) and Pkd1^{fl/fl};XBP1^{fl/fl};Pkhd1-Cre (DKO) mouse models with conditional inactivation of Pkd1 and XBP1 alone or together in the collecting duct were evaluated at P24 by morphological and biochemical parameters: kidney to body weight ratio (KW/BW), cystic index, creatinine, and rates of apoptosis and proliferation. (FIGS. 3A-3B)

Deletion of Pkd1 using Pkhd1-Cre in SKO leads to severe cyst formation at P24 (FIG. 3A). DKO mice display decreased KW/BW as compared to the SKO animals (˜2.7 fold decrease in KW/BW, 0.5±0.1 vs. 1.4±0.2 respectively, ***p<0.001). These changes were accompanied by a ˜1.5-fold decrease in cystic index (42±1.3 vs. 70±1.5, ***p.0.001) and a 2-fold decrease in serum creatinine levels (0.21±0.02 vs. 0.45±0.05, **p<0.01). These effects were XBP1 specific as restoration of active/spliced XBP1 expression via Cre activation of ROSA-flox-stop-XBP1 transgene led to morphological and functional parameters similar to the SKO mice (FIG. 3B).

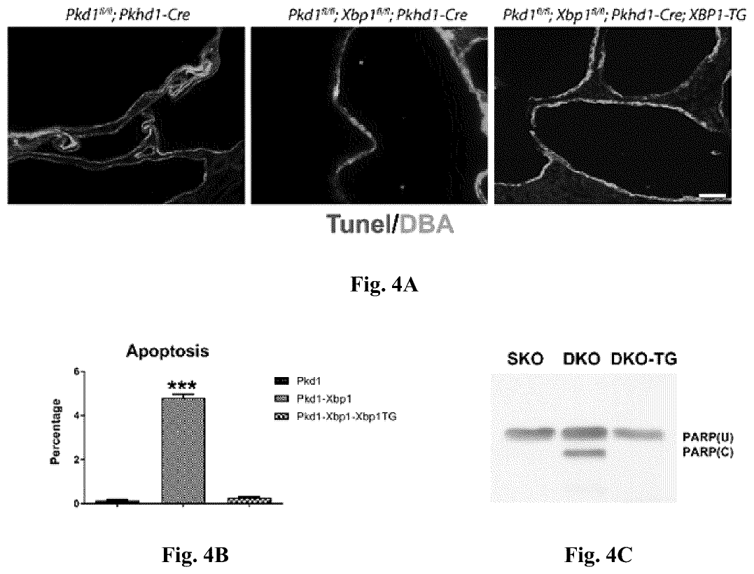

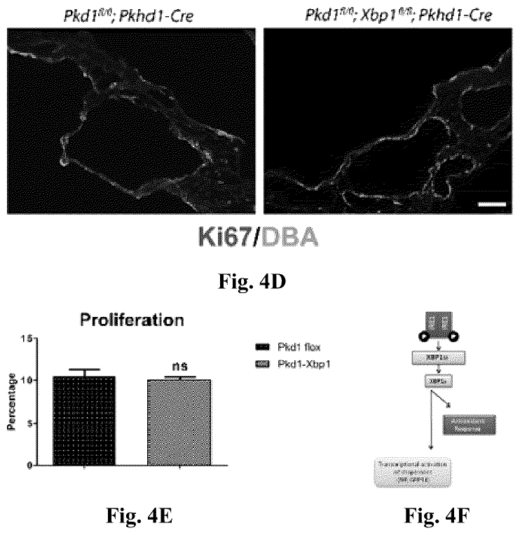

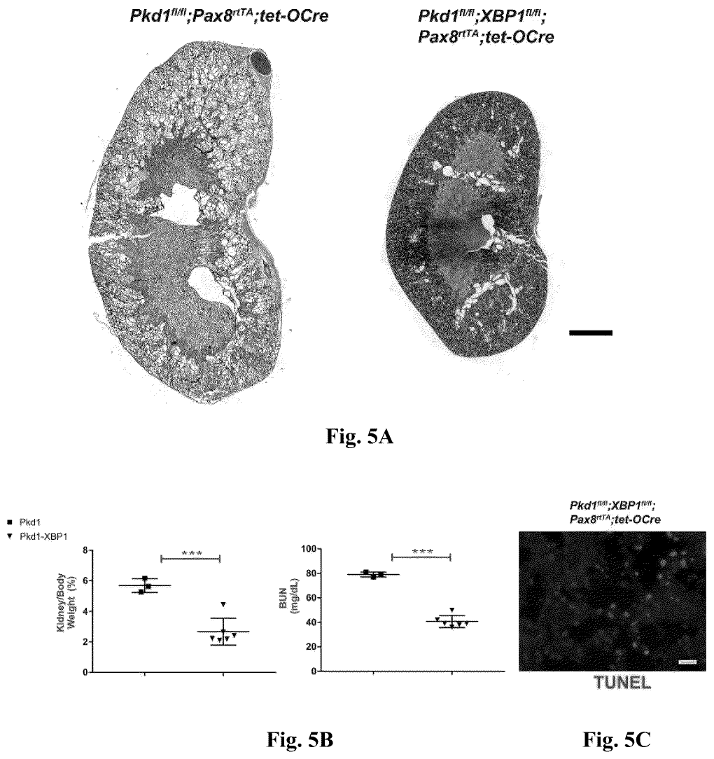

A mechanistic investigation into the effects of XBP1 deletion on a Pkd1 deficient background at P24 in the early Pkhd1-Cre model found that XBP1 inactivation is a potent and specific inducer of apoptosis in cyst cells in vivo (FIGS. 4A-4B). The cystic kidney epithelia in the DKO mice (FIGS. 5A-5B) displayed extensive apoptosis (as seen by TUNEL staining) compared to SKO animals alone (4.7% vs. 0.1%, ***p<0.001; >1000 Dolichos biflorus agglutinin (DBA) cells counted per mouse per genotype) with no changes in proliferation (FIGS. 4D-4E). TUNEL positive cells were confined to collecting ducts (CD) marked by DBA in Pkd1/XBP1 DKO kidneys and were absent from CD of Pkd1 SKO mice and from proximal tubules (PT) of either genotype marked by Lotus tetragonolobus agglutinin (LTA) where Pkhd1-Cre is not active. The increased apoptosis in the Pkd1/XBP1 DKO mice was also confirmed by increased cleaved PARP (FIG. 4C). The pro-apoptotic effect seen on the Pkd1/XBP1 DKO background was reversed to basal Pkd1 SKO levels (both TUNEL and PARP cleavage) when expression of XBP1 was restored in the collecting duct via activation of the ROSA-flox-stop-XBP1 transgene (FIGS. 4A-4C).

The level of active spliced XBP1 was not different between the WT and Pkd1 SKO animals (as seen via RT-PCR), indicating that baseline amounts of spliced XBP1 are important for maintaining the viability of Pkd1 cystic cells in vivo. Furthermore, inactivation of XBP1 alone in the kidney using an embryonic active Cre (KspCdh-Cre) does not have any impact on kidney development/homeostasis or apoptosis.

A PKD model more akin to human disease is the adult model (Ma, et al., 2013, Nat Genet 45:1004-1012) (Pkd1^{fl/fl}; Pax8^{rtTA}; Tet-OCre), where the Cre recombinase is turned on throughout the nephron (except for the PT S3 segment) using a doxycycline inducible system. The mice received doxycycline in the drinking water from P28-P42 to induce Cre and inactivate Pkd1 or Pkd1/XBP1 and were then examined 12 weeks after the end of induction (i.e., 18 weeks of age). As seen in FIGS. 5A-5B, the Pkd1/XBP1 DKO mice display a dramatically lower KW/BW ratio and serum BUN compared with the Pkd1 SKO mice. Similar to the neonatal Pkhd1-Cre driven ADPKD model, double deletion of Pkd1/XBP1 leads to extensive apoptosis of cystic cells (FIG. 5C) with almost no apoptosis seen in the Pkd1 SKO animals.

The effect of IRE1α inhibition was further explored using chemical approaches. Toyocamycin (also known as 4-Aminopyrrolo[2,3-d]pyrimidine-5-carbonitrile 7-(β-D-ribofuranoside), or 7-Deaza-7-cyanoadenosine), is a natural adenosine analog isolated from Streptomyces toyocaensis.

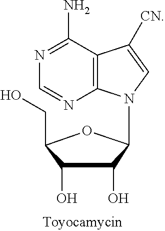

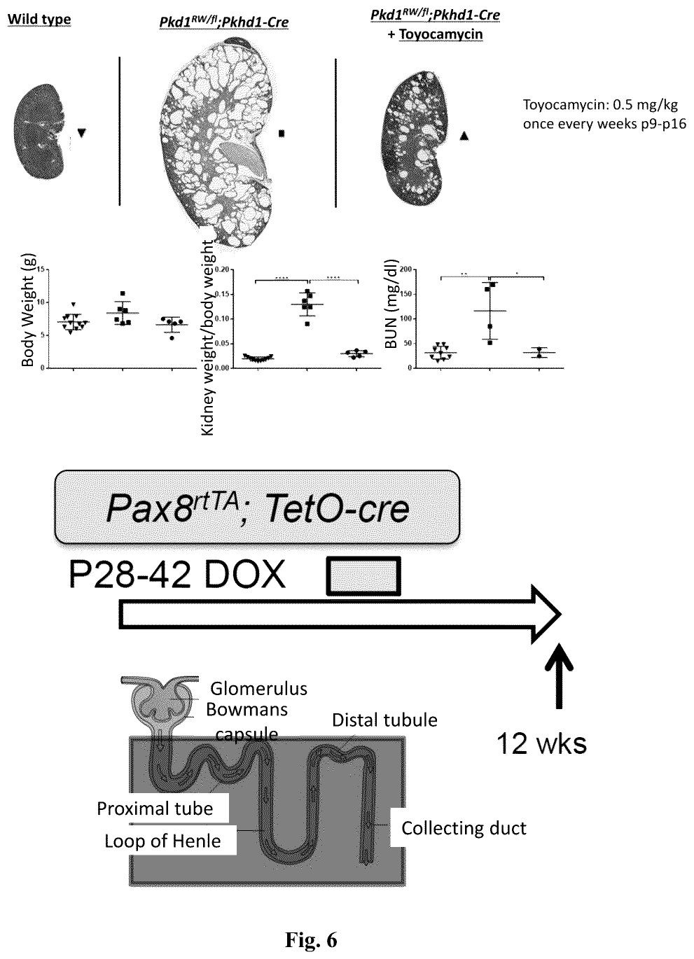

It is a potent inhibitor of IREla endoribonuclease activity, but does not inhibit its kinase activity (Ri, et al., 2012, Blood Cancer J 2:e79). Effect of toyocamycin were investigated in a relevant ADPKD model. For this a mouse model was constructed with a human Pkd1 missense mutation (R2216W) in trans with a floxed allele on a Pkhd1-Cre and Pax8-rtTA; tet-OCre background. As shown in FIG. 6, treatment with toyocamycin from p9 to p16 (once/week) resulted in a dramatic decrease in the KW/BW ratio and BUN (marker of kidney function) in the Pkd1^{RW/fl}; Pkhd1-Cre mice compared with vehicle treated animals.

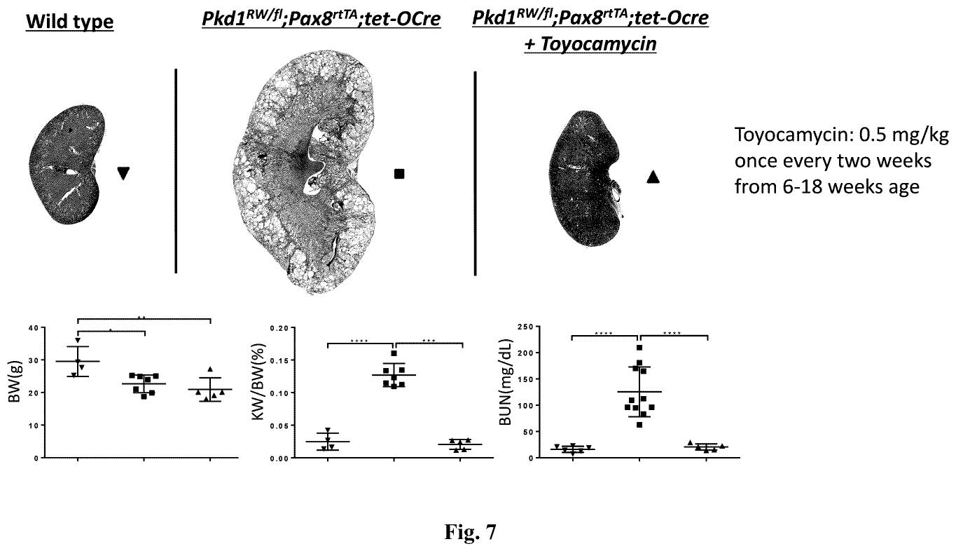

In order to examine whether this drug leads to disease modifying effects in the adult counterpart of this model, Pkd1^{RW/fl}; Pax8^{rtTA}; Tet-OCre mice were generated. In this mice the Cre recombinase is turned on throughout the nephron (except for the PT S3 segment) using a doxycycline inducible system. The mice received doxycycline in the drinking water from P28-P42to induce Cre and inactivate Pkd1 and were then injected with toyocamycin once every 2weeks starting at 6 weeks of age. As seen in FIG. 7, toyocamycin treatment led to an almost complete inhibition of disease progression compared with vehicle treated animals.

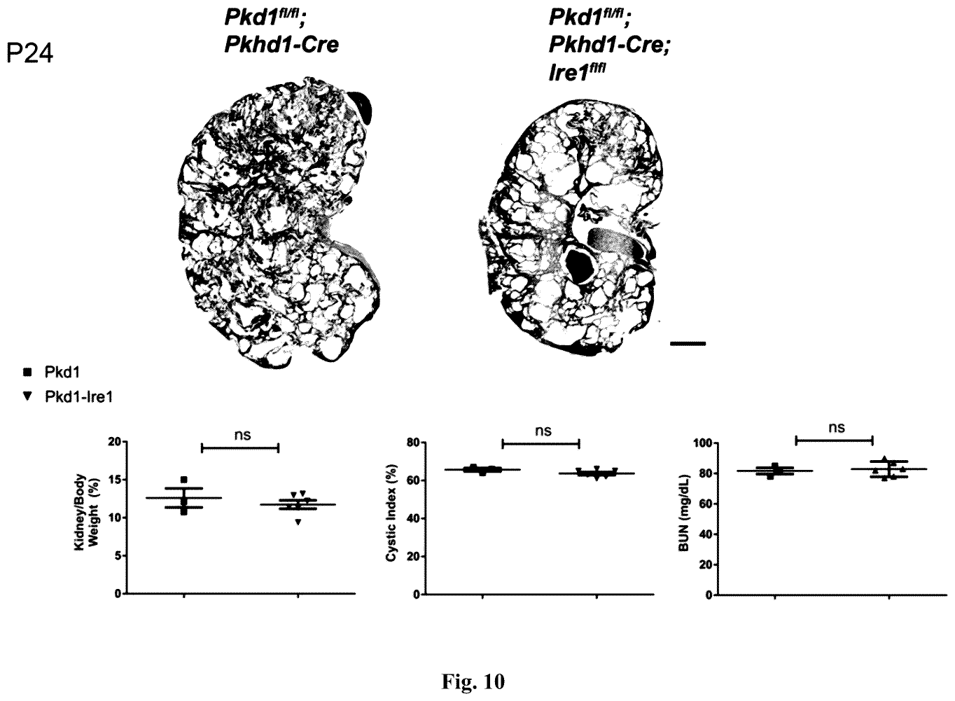

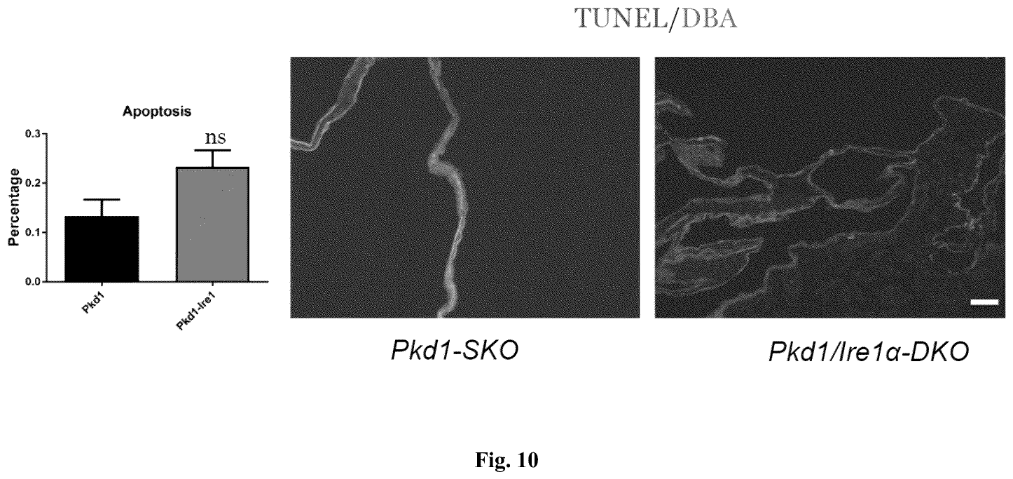

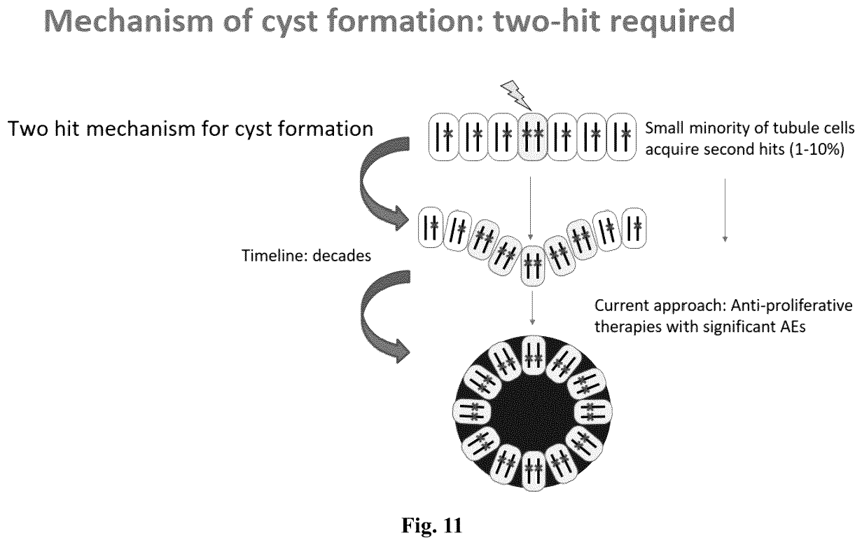

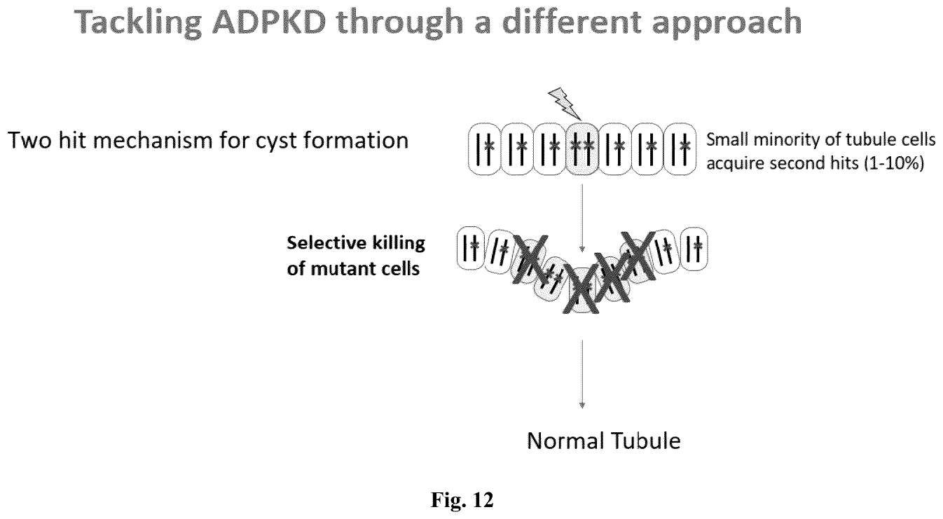

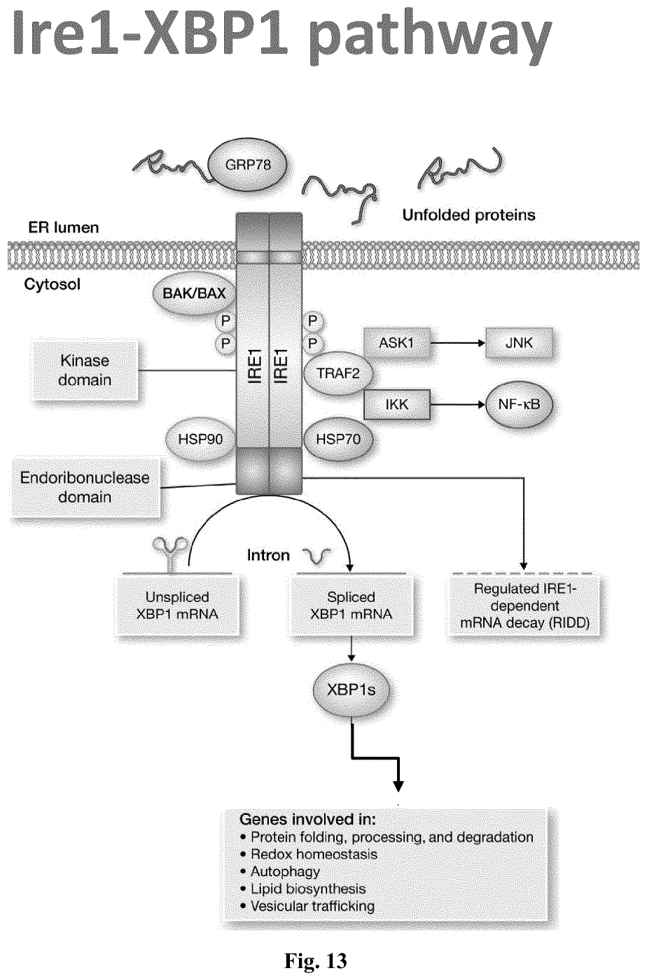

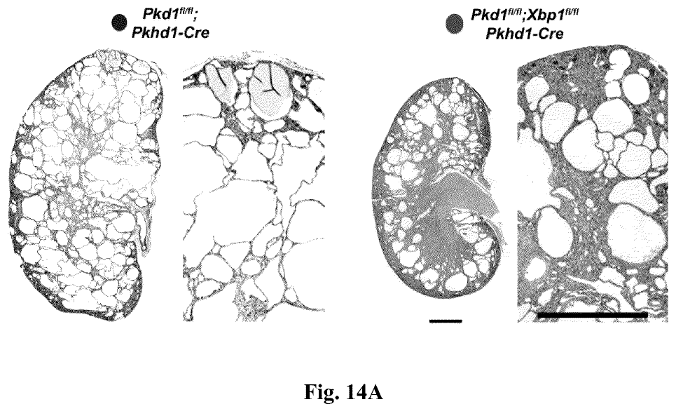

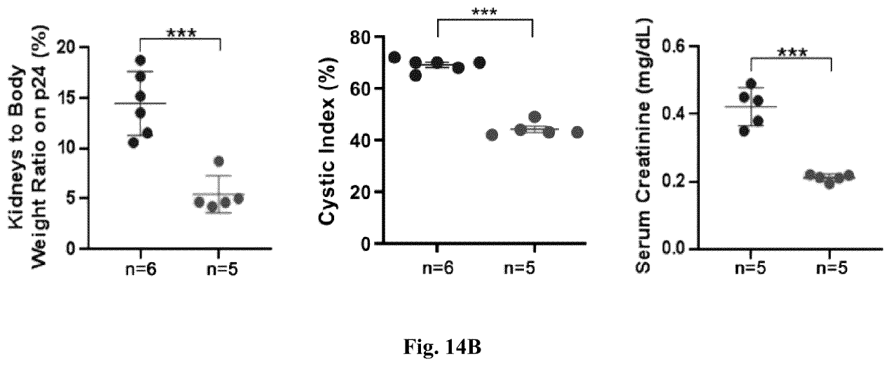

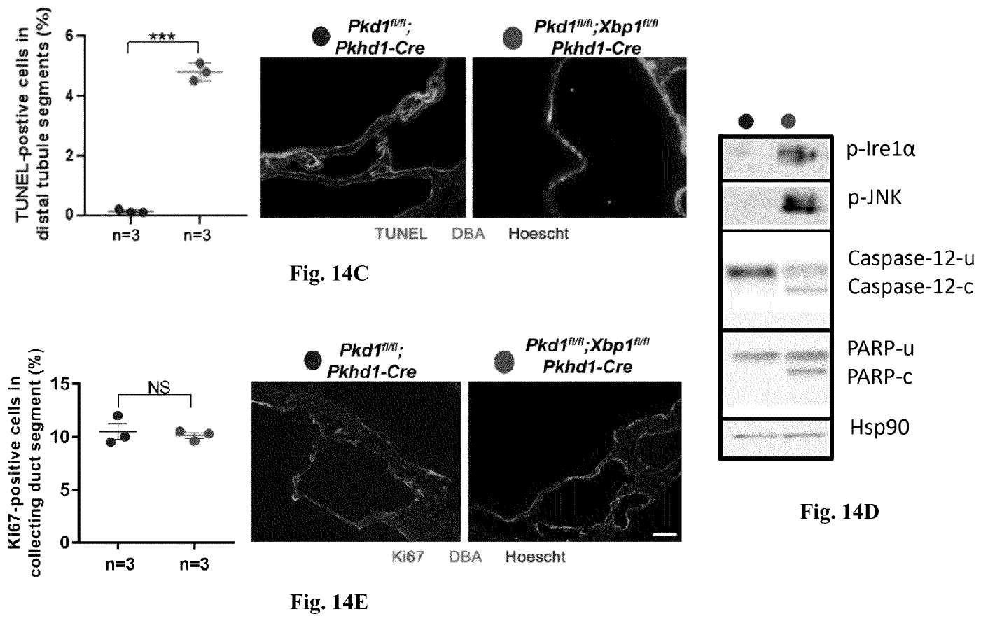

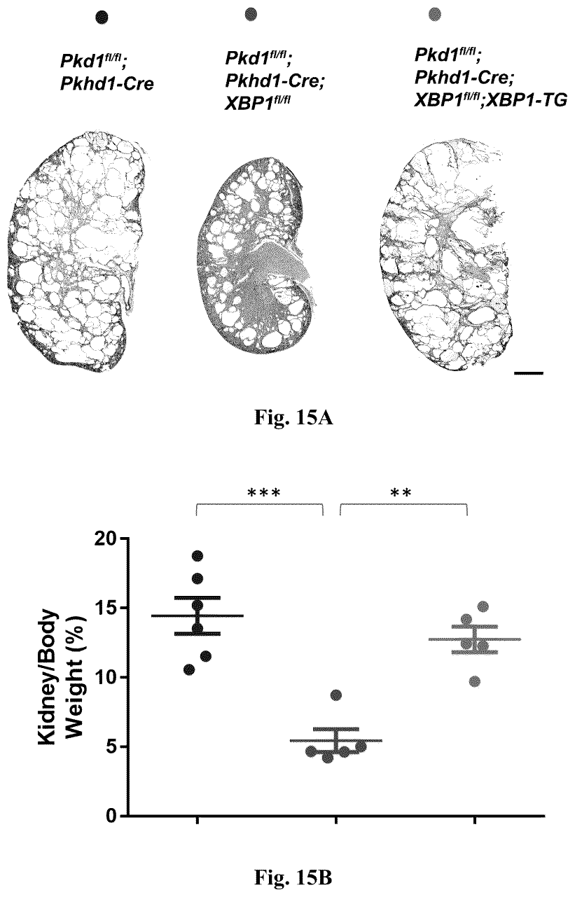

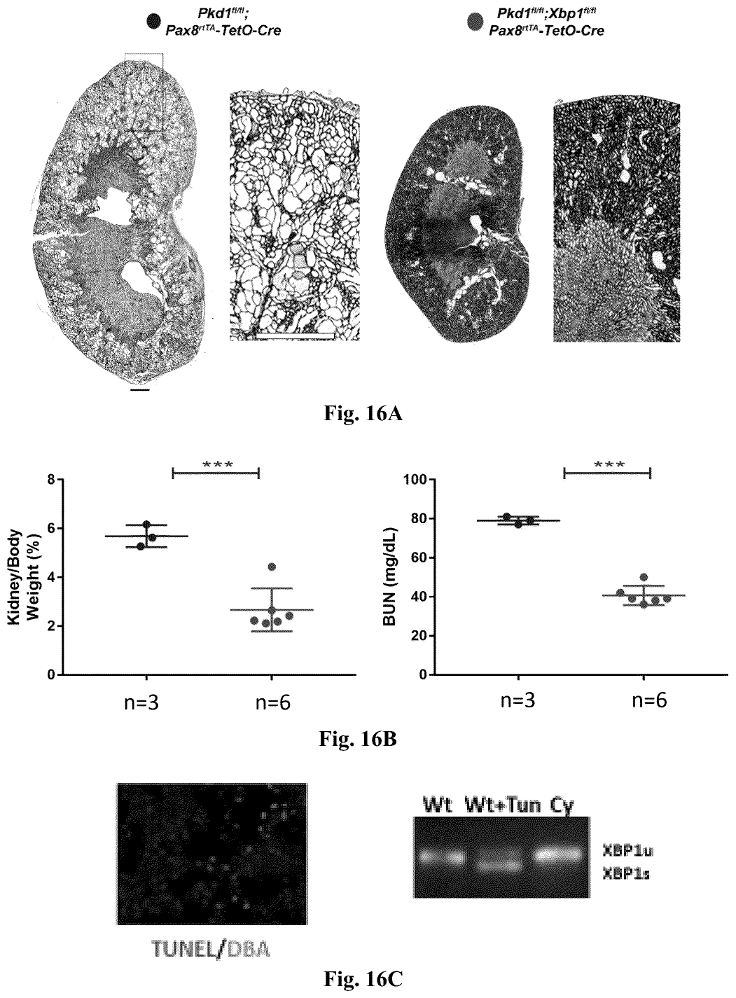

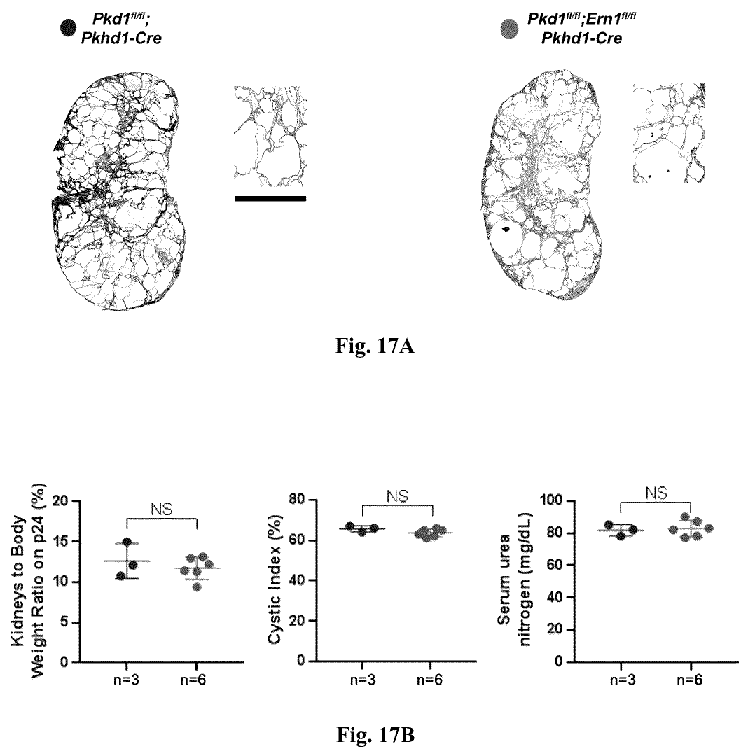

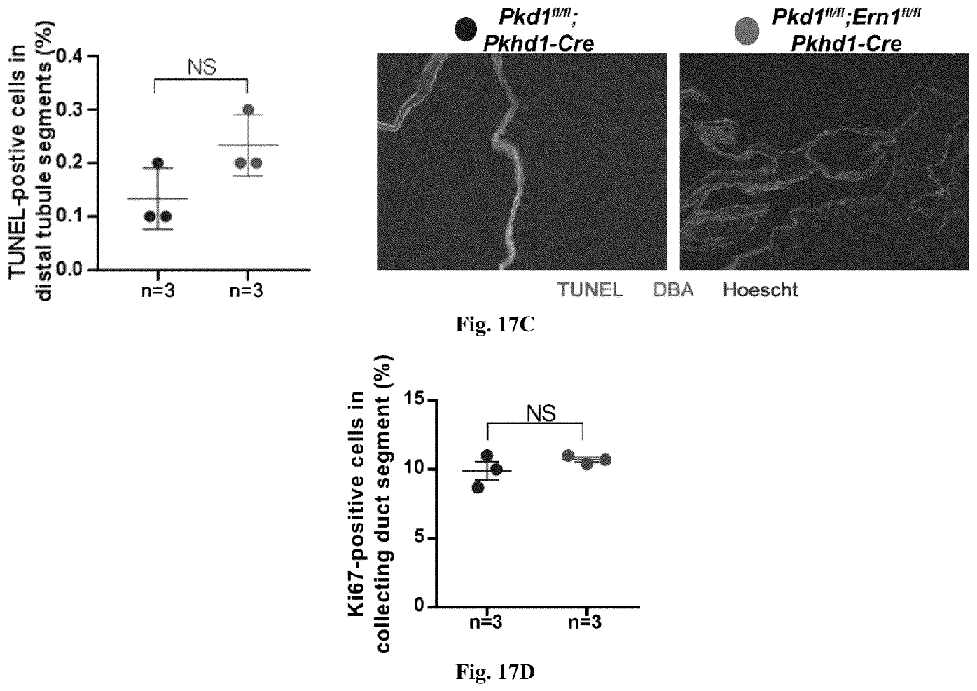

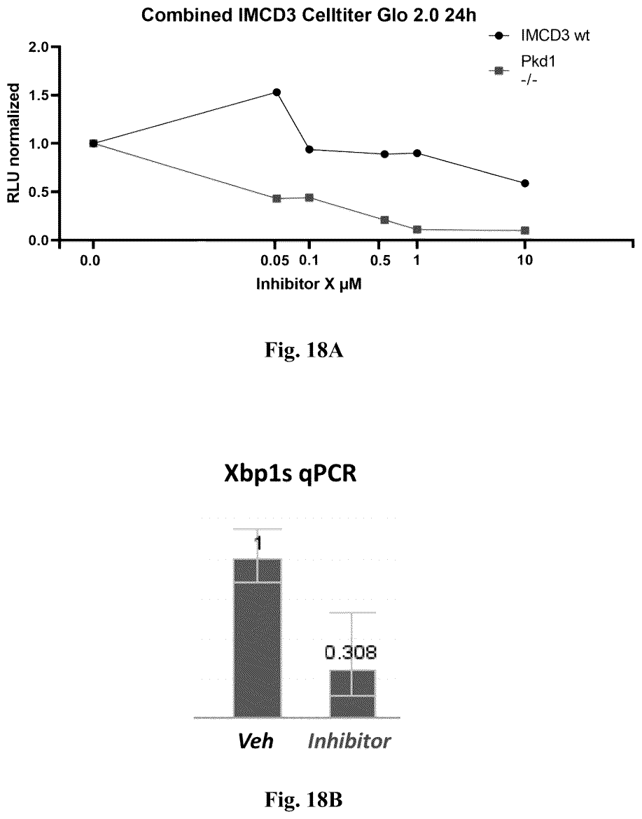

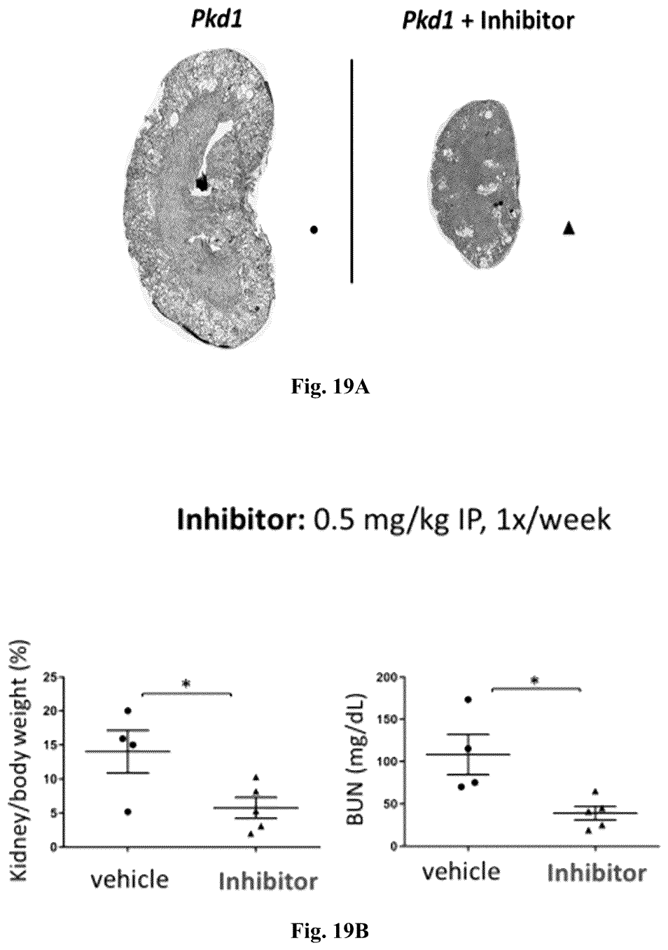

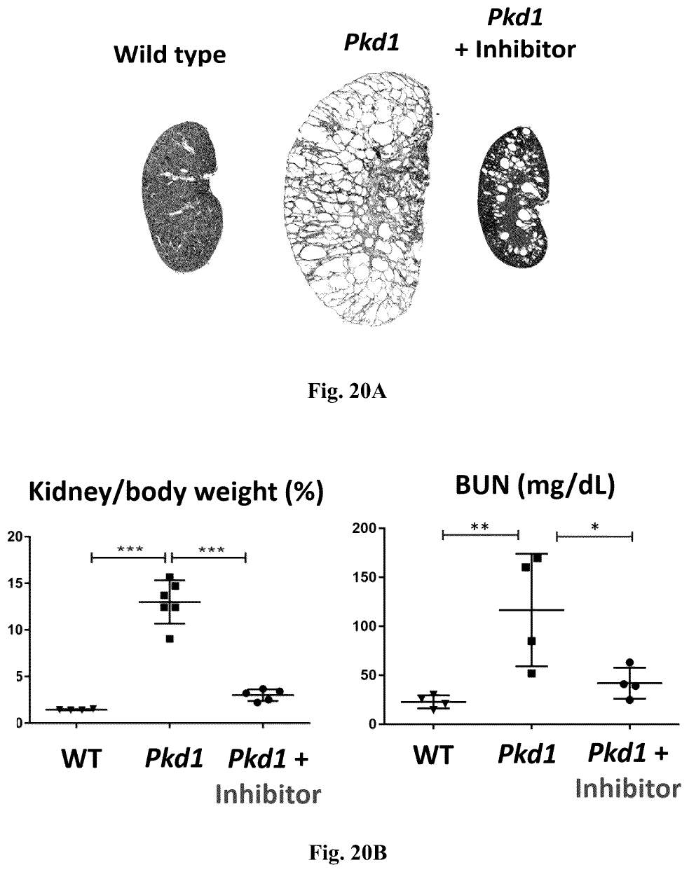

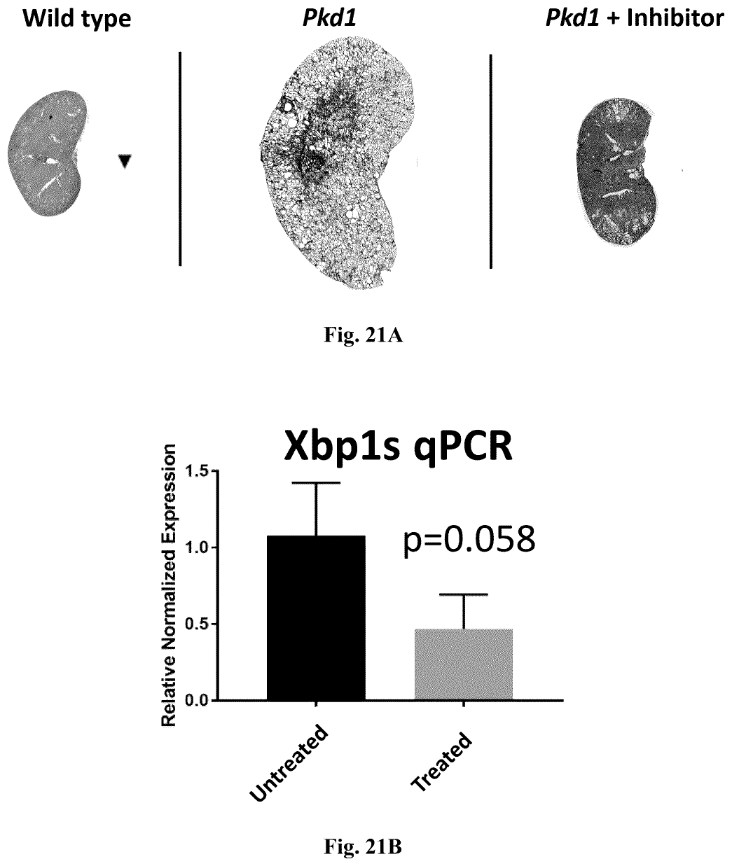

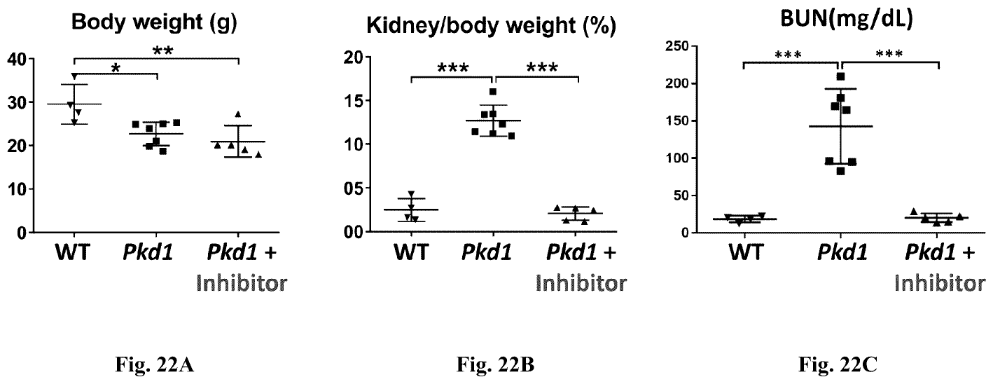

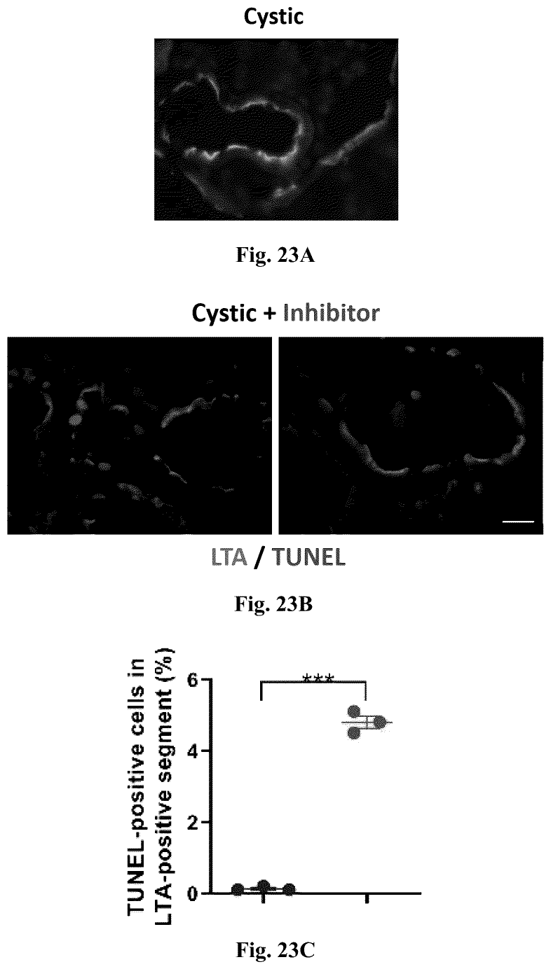

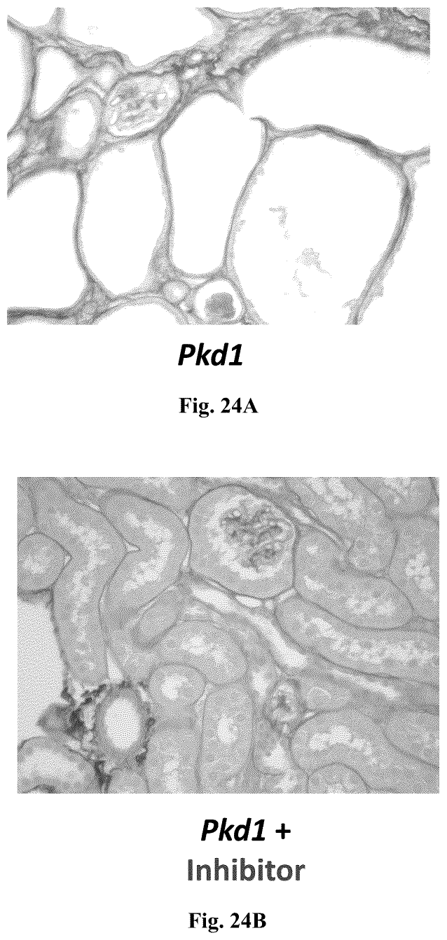

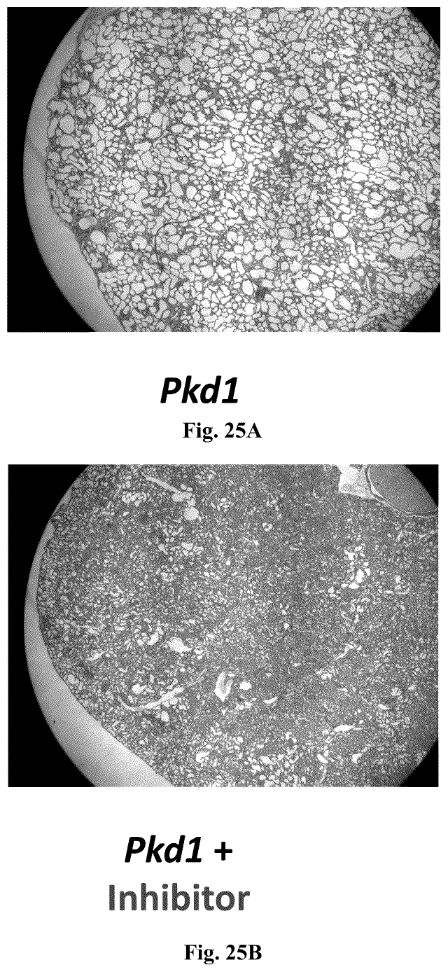

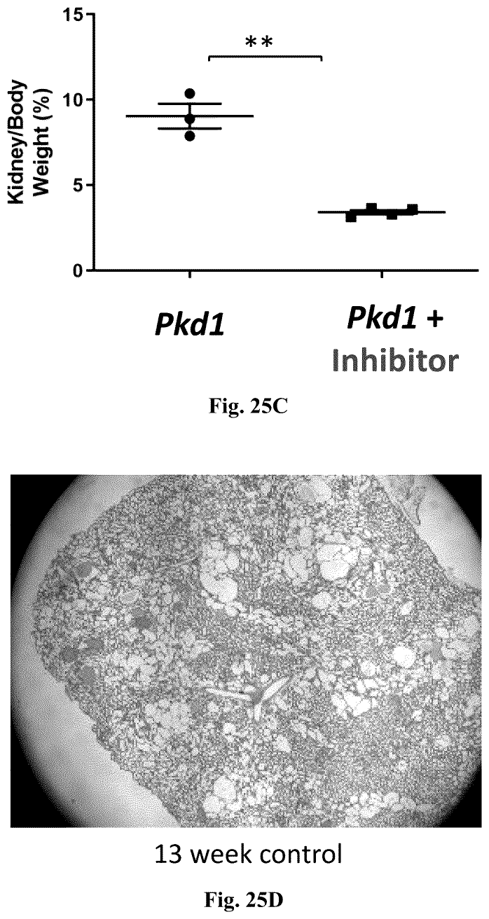

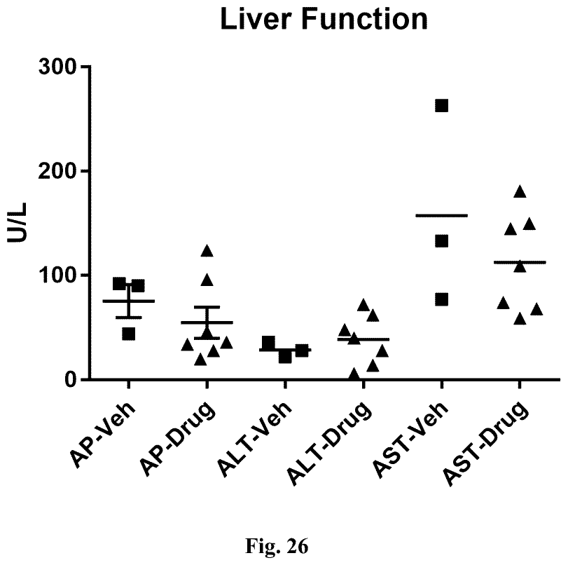

An important finding is that complete inactivation of Ire1α, that includes both its kinase activity and its endoribonuclease activity does not share the therapeutic benefits for ADPKD that selective inhibition of the endoribonuclease activity alone does (FIGS. 9-10). Without wishing to be limited by any theory, the Ire1α kinae activity may be a critical mediator of the apoptotic effect that targets the cyst lining cells. This novel finding further specifies the uniqueness of the therapeutic approach outlined herein.

Overall, the present data show both genetically and pharmacologically that the IRE1α-XBP1 pathway is a novel genetic interactor of Pkd1 and can strongly modulate the progression of ADPKD in murine models by protecting Pkd1 kidney cyst cells from apoptosis without impacting their proliferation. Tilting the balance from low to high apoptosis levels (via inactivation of XBP1 on a Pkd1 KO background) given similar proliferation profiles provides a viable therapeutic option in the context of cystic kidney disease. Given that the target pathway in this case is very well characterized and not needed for kidney development or homeostasis, the present data offer a therapeutic option for slowing down ADPKD (and possibly ARPKD and ADPLD) by targeting IRE1α-XBP1. Furthermore, the present studies identified toyocamycin as a potent agent that leads to a dramatic decrease in polycystic kidney disease progression in both early and adult mouse models. This agent can be used for further pre-clinical/clinical development for the treatment of ADPKD.

In certain embodiments, the present invention provides a composition for treating ADPKD and/or ADPLD and/or ARPKD in a subject, wherein the composition comprises an IRE1α endoribonuclease activity inhibitor.

In certain embodiments, the IRE1α endoribonuclease activity inhibitor comprises a peptide, antibody, small molecule, antagonist, aptamer, or peptidomimetic, and any combinations thereof, that reduces IRE1α endoribonuclease activity.

In certain embodiments, the IRE1α endoribonuclease activity inhibitor comprises toyocamycin, or a salt, solvate, tautomer, geometric isomer, enantiomer, or diastereoisomer thereof:

In certain embodiments, small molecules or peptidomimetics contemplated herein are prepared as prodrugs. A prodrug is an agent converted into the parent drug in vivo. In one embodiment, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound. Prodrugs are known to those skilled in the art, and may be prepared using methodology described in the art.

The small molecule and small molecule compounds described herein may be present as salts even if salts are not depicted and it is understood that the invention embraces all salts, prodrugs and solvates of the inhibitors depicted here, as well as the non-salt and non-solvate form of the inhibitors, as is well understood by the skilled artisan. In some embodiments, the salts of the inhibitors of the invention are pharmaceutically acceptable salts.

Where tautomeric forms may be present for any of the inhibitors described herein, each and every tautomeric form is intended to be included in the present invention, even though only one or some of the tautomeric forms may be explicitly depicted.

The invention also includes any or all of the stereochemical forms, including any enantiomeric or diastereoisomeric forms of the inhibitors described. The recitation of the structure or name herein is intended to embrace all possible stereoisomers of inhibitors depicted. All forms of the inhibitors are also embraced by the invention, such as crystalline or non-crystalline forms of the inhibitors. Compositions comprising an inhibitor of the invention are also intended, such as a composition of substantially pure inhibitor, including a specific stereochemical form thereof, or a composition comprising mixtures of inhibitors of the invention in any ratio, including two or more stereochemical forms, such as in a racemic or non-racemic mixture.

In certain embodiments, the small molecule inhibitor of the invention comprises an analog or derivative of an inhibitor described herein. In other embodiments, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, the isotope comprises deuterium. In certain embodiments, isotopically labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In certain embodiments, the pharmaceutical composition is coformulated with at least one additional agent that treats or prevents ADPKD and/or ADPLD in a mammal.

### Salts

The compounds described herein may form salts with acids or bases, and such salts are included in the present invention. The term “salts” embraces addition salts of free acids or bases that are useful within the methods of the invention. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the invention.

Suitable pharmaceutically acceptable base addition salts of compounds of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Methods

The invention provides a method of treating or preventing Autosomal Dominant Polycystic Kidney Disease (ADPKD) and/or Autosomal Dominant Polycystic Liver Disease (ADPLD) and/or Autosomal Recessive Polycystic Kidney Disease (ARPKD) in a mammal.

In certain embodiments, the method comprises administering to the mammal a therapeutically effective amount of an IRE1α endonuclease activity inhibitor. Examples of IRE1α endonuclease activity inhibitors includes 4μ8C (7-Hydroxy-4-methyl-2-oxo-2H-1-benzopyran-8-carboxaldehyde), STF 083010 (N-[(2-Hydroxy-1-naphthalenyl)methylene]-2-thiophenesulfonamide), MKC8866 (CAS #1338934-59-0), Kira 6 (CAS #1589527-65-0), Kira 8 (CAS #1630086-20-2), MKC3946 (CAS #1093119-54-0), GSK2850163 (CAS #2121989-91-9), 6-Bromo-2-hydroxy-3-methoxybenzaldehyde (CAS #20035-41-0), 3-methoxy-6-bromosalicylaldehyde salicylaldimines, toyocamycin, N9-(3-(dimethylamino)propyl)-N3,N3,N6,N6-tetramethylacridine-3,6,9-triamine (3,6-DMAD), Hydroxy-aryl-aldehydes (HAA), irestatin, and the like. Examples of IRE1α endonuclease activity inhibitors are described in, for example, US 2017/0165259 A1, US 2019/0084989 A1, US 2019/0314330 A1, and US 2020/0024247 A1. These references are hereby incorporated herein by reference.

In certain embodiments, the inhibitor does not inhibit IRE1α kinase activity.

In certain embodiments, the inhibitor comprises a peptide, antibody, small molecule, antagonist, aptamer, or peptidomimetic, and any combinations thereof.

In certain embodiments, the small molecule comprises toyocamycin, or a salt, solvate, tautomer, geometric isomer, enantiomer, and/or diastereoisomer thereof.

In certain embodiments, the administration slows or inhibits development of a cyst in the mammal's kidney or liver.

In certain embodiments, the inhibitor is the only therapeutically effective agent administered to the mammal.

In certain embodiments, the inhibitor is the only therapeutically effective agent administered to the mammal in an amount sufficient to treat or prevent ADPKD and/or ADPLD and/or ARPKD in the mammal.

In certain embodiments, the mammal is further administered at least one additional agent that treats or prevents ADPKD and/or ADPLD and/or ARPKD.

In certain embodiments, the at least one additional agent is Tolvaptan, or a salt, solvate, tautomer, geometric isomer, enantiomer, and/or diastereoisomer thereof.

In certain embodiments, the mammal is human.

In certain embodiments, the inhibitor is administered to the mammal by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous routes.

### Administration/Dosage/Formulations

The regimen of administration may affect what constitutes an effective amount. The therapeutic formulations may be administered to the subject either prior to or after the onset of a disease or disorder contemplated in the invention. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. The pharmaceutical compositions useful for practicing the invention may be administered to deliver a dose of from 0.01 ng/kg/day and 100 mg/kg/day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated in the invention.

In one embodiment, the compositions of the invention are formulated using one or more pharmaceutically acceptable excipients or carriers. In one embodiment, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the invention are administered to the patient in dosages that range from one to five times per day or more. In another embodiment, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, and any and all whole or partial increments therebetween.

In some embodiments, the dose of a compound of the invention is from about 1 mg and about 2,500 mg, and any and all whole or partial increments thereof.

In one embodiment, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. patent applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In one embodiment, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of a disease or disorder contemplated in the invention. The skilled artisan is able to determine appropriate dosages depending on these and other factors.

A suitable dose of a compound of the present invention may be in the range of from about 0.01 mg to about 5,000 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In one embodiment, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. The data obtained from cell culture assays and animal studies are optionally used in formulating a range of dosage for use in human. The dosage of such compounds lies preferably within a range of circulating concentrations that include the ED50 with minimal toxicity. The dosage optionally varies within this range depending upon the dosage form employed and the route of administration utilized.

### Combination Therapies

In certain embodiments, the compounds of the invention are useful in the methods of the invention in combination with at least one additional agent useful for treating or preventing a disease or disorder contemplated herein. This additional agent may comprise compounds identified herein or compounds, e.g., commercially available compounds, known to treat, prevent or reduce the symptoms of a disease or disorder contemplated herein.

A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Kits

The invention includes a kit comprising at least one compound contemplated herein, an applicator, and an instructional material for use thereof. The instructional material included in the kit comprises instructions for preventing or treating a disease or disorder contemplated herein in a mammal. The instructional material recites the amount of, and frequency with which, the at least one compound contemplated herein should be administered to the mammal. In other embodiments, the kit further comprises at least one additional agent that prevents or treats a disease or disorder contemplated herein in a mammal.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. As used herein, each of the following terms has the meaning associated with it in this section.

Generally, the nomenclature used herein and the laboratory procedures in cell culture, molecular genetics, pharmacology and organic chemistry are those well-known and commonly employed in the art.

Standard techniques are used for biochemical and/or biological manipulations. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, NY, and Ausubel et al., 2002, Current Protocols in Molecular Biology, John Wiley & Sons, NY), which are provided throughout this document.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A disease or disorder is “alleviated” if the severity or frequency of at least one sign or symptom of the disease or disorder experienced by a patient is reduced.

As used herein, the terms “analog,” “analogue,” or “derivative” are meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically.

As used herein, the term “binding” refers to the adherence of molecules to one another, such as, but not limited to, enzymes to substrates, antibodies to antigens, DNA strands to their complementary strands. Binding occurs because the shape and chemical nature of parts of the molecule surfaces are complementary. A common metaphor is the “lock-and-key” used to describe how enzymes fit around their substrate. A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

“Naturally occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” or “therapeutically acceptable salt” refers to a salt of the administered compounds prepared from pharmaceutically acceptable non-toxic acids, including inorganic acids or bases, organic acids or bases, solvates, hydrates, or clathrates thereof.

The terms “pharmaceutically effective amount” and “effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

As used herein, the terms “polypeptide,” “protein” and “peptide” are used interchangeably and refer to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

By the term “specifically binds,” as used herein, is meant a molecule, such as an antibody, which recognizes and binds to another molecule or feature, but does not substantially recognize or bind other molecules or features in a sample.

A “therapeutic” treatment is a treatment administered to a subject who exhibits signs of pathology, for the purpose of diminishing or eliminating those signs.

As used herein, the term “therapeutically effective amount” is an amount of a compound of the invention, that when administered to a patient, ameliorates a symptom of the disease or disorder. The amount of a compound of the invention that constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

As used herein, “treating a disease or disorder” means reducing the frequency with which a symptom of the disease or disorder is experienced by a patient. Disease and disorder are used interchangeably herein.

As used herein, the term “treatment” or “treating” encompasses prophylaxis and/or therapy. Accordingly the compositions and methods of the present invention are not limited to therapeutic applications and can be used in prophylaxis ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms. As used herein, the term “wild-type” refers to the genotype and phenotype that is characteristic of most of the members of a species occurring naturally and contrasting with the genotype and phenotype of a mutant.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

FIG. 1 illustrates a diagram view of the unfolded protein response with the three effector pathways: IRE1α, PERK, and ATF6.

FIG. 2 illustrates a schematic depiction of Irela dependent pathways, i.e. acting either via XBP1 in order to increase adaptation and recovery under stress or Ire1α-driven XBP1-independent to trigger a pro-apoptotic response under circumstances of unresolved chronic stress.

FIGS. 3A-3B illustrate the finding that Pkd1 and Xbp1 interact at the genetic level, and active Xbp1 is not upregulated from baseline in the absence of Pkd1. FIG. 3A: Inactivation of XBP1 can slow down progression of polycystic kidney disease in an early ADPKD model due to inactivation of polycystin-1 (middle panel). This effect is specific to XBP1 as restoration of its expression in the DKO animals via a ROSA-flox-stop transgene (right panel) leads to similar progression of disease as seen in the SKO mice (left panel). FIG. 3B: Morphological (KW/BW ratio, cystic index) and functional parameters (serum creatinine) are significantly improved on the Pkd1/XBP1 DKO background vs. Pkd1 single knockout background. n=6,5,4.

FIGS. 4A-4F illustrate the finding that inactivation of XBP1 induces apoptosis specifically in kidney segments where Pkd1 was inactivated (Pkd1 cystic epithelia). FIG. 4A: The presence of apoptosis was examined by TUNEL staining on cryosections from Pkd1^{fl/fl};Pkhd1-Cre (SKO), Pkd1^{fl/fl};Xbp1^{fl/fl};Pkhd1-Cre (DKO), or DKO mice expressing a ROSA-floxstop-XBP1 transgene (DKO-TG). FIG. 4B: Quantification of the apoptotic rates examined in panel A. FIG. 4C: PARP cleavage is increased in the DKO animals compared to either SKO or DKO+XBP1-TG. FIGS. 4D-4E: Proliferation rates as assessed by Ki67 staining were not different between SKO vs. DKO animals.

FIGS. 5A-5C illustrate the finding that Xbp1 inactivation leads to apoptosis of Pkd1 null cells in an adult ADPKD model. FIG. 5A: Inactivation of XBP1 in an adult ADPKD model (Pkd1^{fl/fl}, Pax8^{rtTA}; tet-OCre) leads to a dramatic improvement in the cystic phenotype. FIG. 5B: This effect is recapitulated by an improvement in kidney to body weight ratio and serum BUN. FIG. 5C: The presence of apoptosis was observed by TUNEL staining on cryosections from Pkd1^{fl/fl};XBP1^{fl/fl};Pax8^{rtTA};tet-OCre animals. No apoptosis was seen in the Pkd1^{fl/fl};Pax8^{rtTA};tet-OCre animals (data not shown). n=3,6.

FIG. 6 illustrates the finding that toyocamycin treatment in Pkd1^{RW/fl}; Pkhd1-Cre mice leads to a dramatic improvement in the cystic phenotype as seen by morphological (top panel) and functional (bottom panel) parameters.

FIG. 7 illustrates the finding that toyocamycin treatment in Pkd1^{RW/fl}; Pax8^{rtTA}; tet-OCre mice leads to a dramatic improvement in the cystic phenotype as seen by morphological (top panel) and functional (bottom panel) parameters.

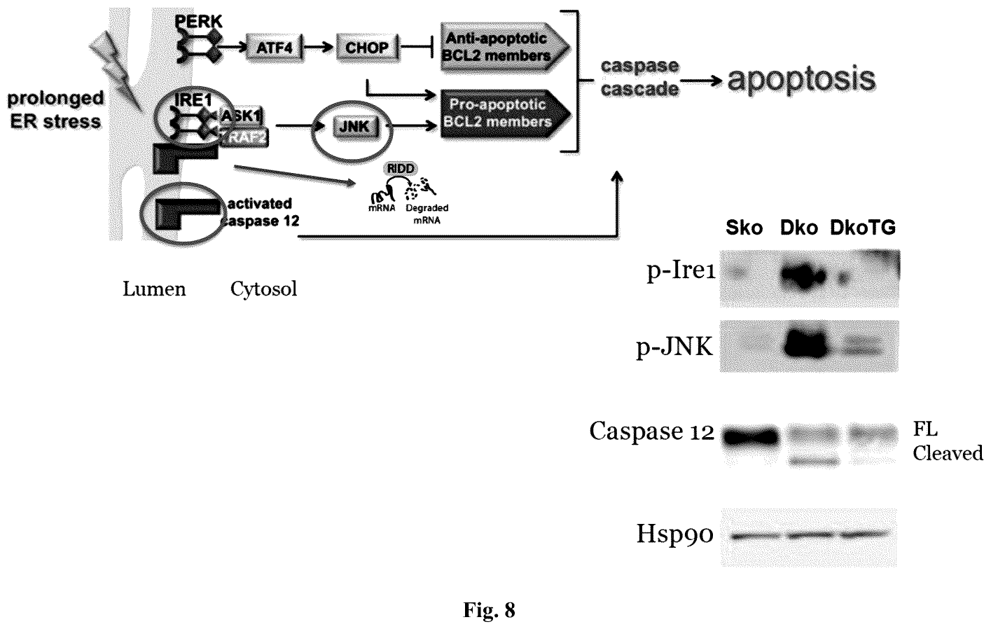

FIG. 8 illustrates the Ire1α-dependent cells death pathway, as well as the finding the Ire1α is activated in the Pkd1/Xbp1 DKO mice.

FIG. 9 illustrates the finding that Ire1α inactivation does not impact ADPKD progression in absence of Pkd1.

FIG. 10 illustrates the finding that apoptosis is not impacted in the Pkd1-Ire1α DKO mice. This demonstrates that the Ire1α-dependent pro-apoptosis function drives the beneficial effect on the Pkd1-Xbp1 DKO background. In certain non-limiting embodiments, inhibitors of Ire1α endoribonuclease function, which are not Ire1α kinase inhibitors, have beneficial effects in ADPKD models.

### Example 2

Based on the common involvement of PC1 and Ireα-XBP1 in proliferative neoplastic or malignant backgrounds, the present study investigated whether Ireα-XBP1 can modulate ADPKD progression via a direct genetic interaction with Pkd1. Furthermore, it was hypothesized that the Ireα-XBP1 pathway may provide a survival advantage to mutant cells within the hypoxic cystic microenvironment and its inhibition would result in a specific viability defect towards cystic cells leading to improved disease progression.

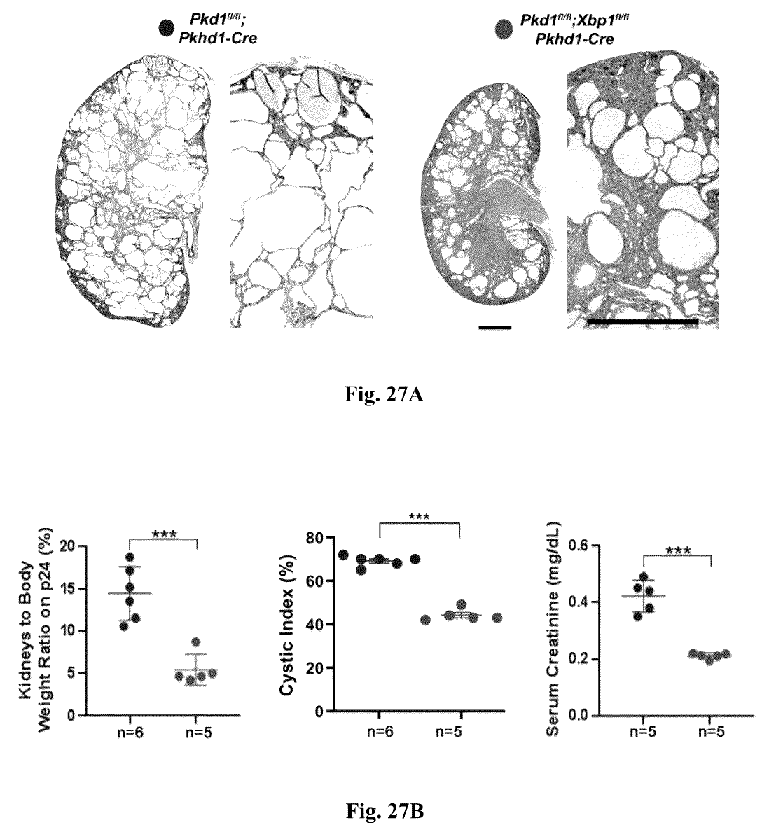

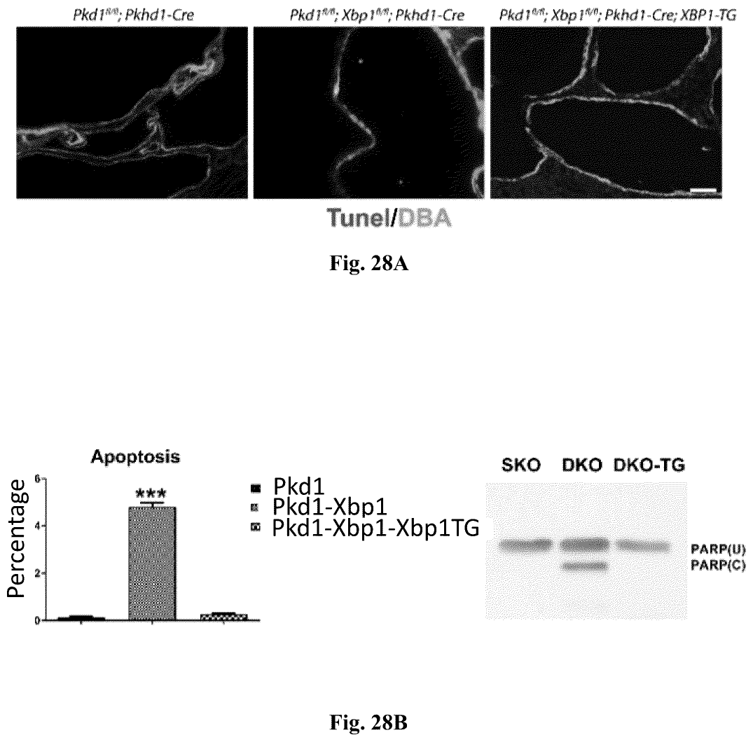

The present study used relevant murine models of kidney Pkd1, XBP1, and Ire1α inactivation to test their genetic interaction. To this end, Pkd1^{fl/fl}, Pkhd1-Cre (SKO) and Pkd1^{fl/fl};XBP1^{fl/fl};Pkhd1-Cre (DKO) mouse models with conditional inactivation of Pkd1 and XBP1 alone or together in the collecting duct were evaluated at P24 by morphological and biochemical parameters: kidney to body weight ratio (KW/BW), cystic index, creatinine, and rates of apoptosis. Deletion of Pkd1 using Pkhd1-Cre in SKO leads to severe cyst formation at P24 (FIGS. 27A-27B). DKO mice display decreased KW/BW as compared to the SKO animals (˜2.7 fold decrease in KW/BW, ***p<0.001). These changes were accompanied by a ˜1.5-fold decrease in cystic index (***p<0.001) and a 2-fold decrease in serum creatinine levels (***p<0.001) (FIGS. 28A-28B).

The cystic kidney epithelia in the DKO mice (FIGS. 28A-28B) displayed extensive apoptosis compared to SKO animals alone (***p<0.001) with no changes in proliferation (data not shown). To further validate this result, the present study found that the levels of cleaved PARP were higher in the DKO vs. SKO mice (FIG. 28C). This effect was cell autonomous as re-expression of XBP1 via the Rosa-floxstop-TG 28 leads to the disappearance of the apoptotic phenotype seen in the DKO mice (FIGS. 28A-28C) and a reversal of the cystic phenotype to a similar severity as that seen in SKO mice (data not shown).

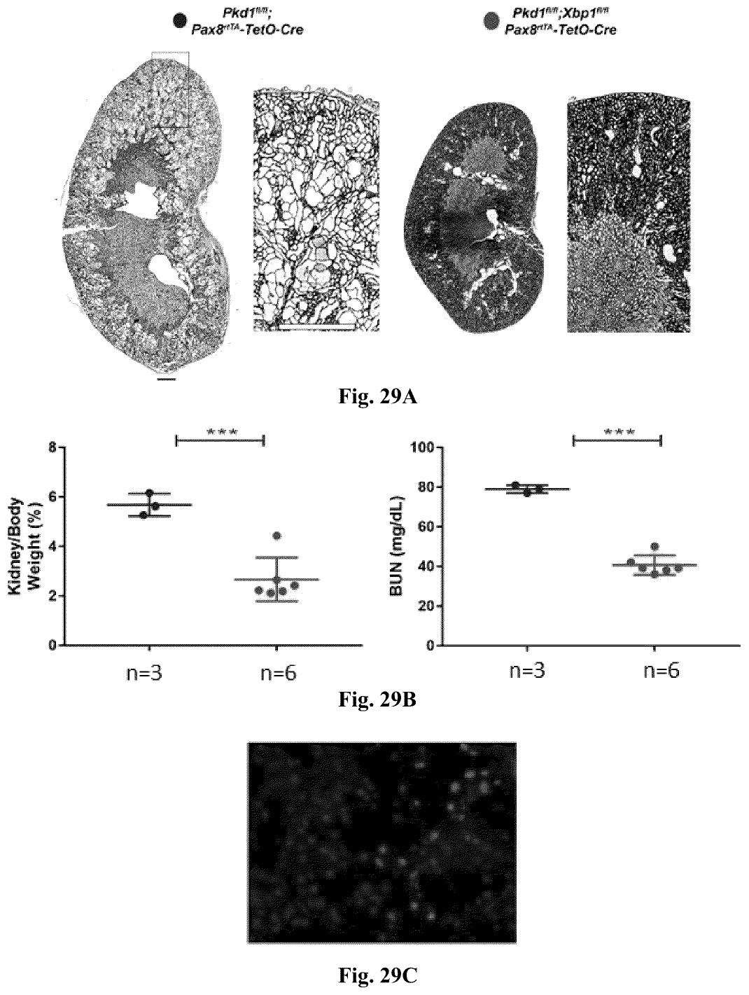

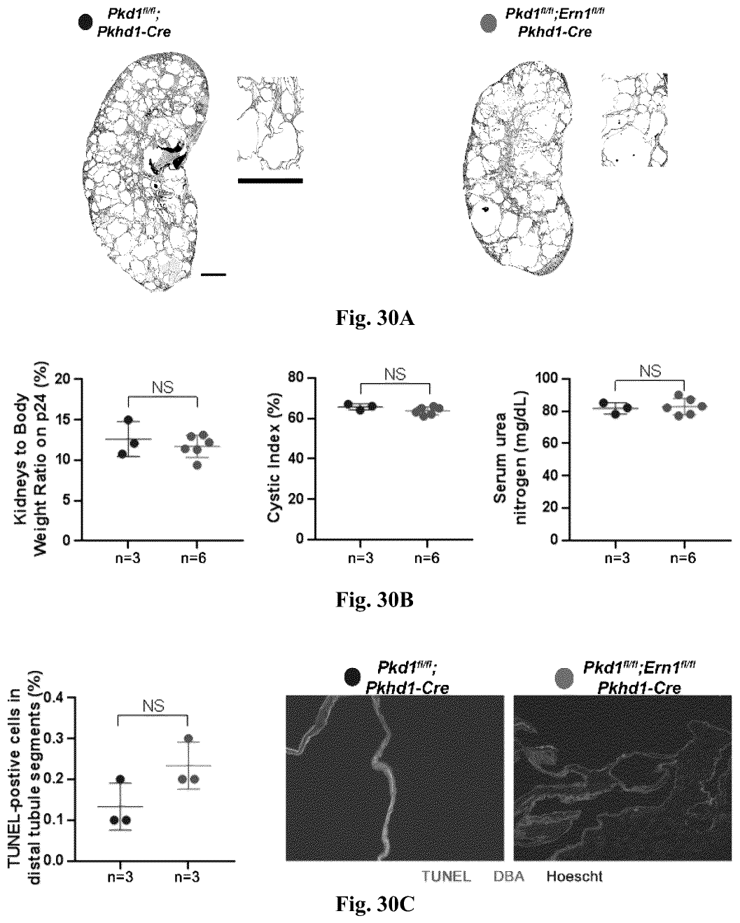

The apoptotic effect due to XBP1 inactivation is Pkd1 deletion specific as XBP1 inactivation in the kidney at embryonic day 10.5 using Ksp-Cre does not cause any apoptosis nor impact kidney development as seen at post developmental day 30. Interestingly, the level of active spliced XBP1 expression was not different between the WT and Pkd1 KO kidneys (data not shown) indicating that baseline levels of spliced XBP1 are involved in maintaining the viability of Pkd1 cystic cells in vivo. The present study investigated whether the beneficial effect of XBP1 inactivation can impact disease progression in an adult, slow-progressing PKD model more akin to the human disease. To this end, Pkd1^{flox/flox}; Pax8rtTA; tet-OCre (SKO) or Pkd1^{flox/flox};XBP1^{flox/flox}; Pax8rtTA; tet-OCre (DKO) mice were generated. For this model, mice are given doxycycline water between P28 and P42 which leads to activation of Cre throughout the nephron (except the S3 segment of the proximal tubule) under the control of the Pax8promoter. Following Cre activation, mice were aged for 12 weeks and sacrificed at 18 weeks of age. As seen in FIGS. 29A-29C, at 18 weeks of age the control mice display a severe cystic phenotype. Gratifyingly, similar to the neonatal model, concomitant inactivation of Pkd1 and XBP1 leads to a significant improvement in the kidney to body weight ratio (***p<0.001) and BUN (***p<0.001). Furthermore, apoptosis of cyst lining epithelia is seen in the Pkd1/XBP1 DKO mice (FIG. 29C). In order to further dissect the role of Ire1α as a regulator of the cystic phenotype in the absence of XBP1, the present study inactivated Ire1α (using Pkhd1-Cre) concomitantly with Pkd1. Intriguingly, no impact on the cystic phenotype was observed between Pkd1 vs. Pkd1/Ire1α animals as assessed via morphological and functional parameters (KWBW ratio, cystic index, and BUN; p>0.05; FIGS. 30A-30B).

Furthermore, no difference in apoptosis was seen on the Pkd1/Ire1α vs Pkd1 single knockout background (FIG. 30C). These genetic data implicate the Ire1α-XBP1 pathway in the pathogenesis of ADPKD via a specific apoptotic response of cystic cells likely driven by the kinase function of Ire1α (which is lost when the whole protein is impaired).

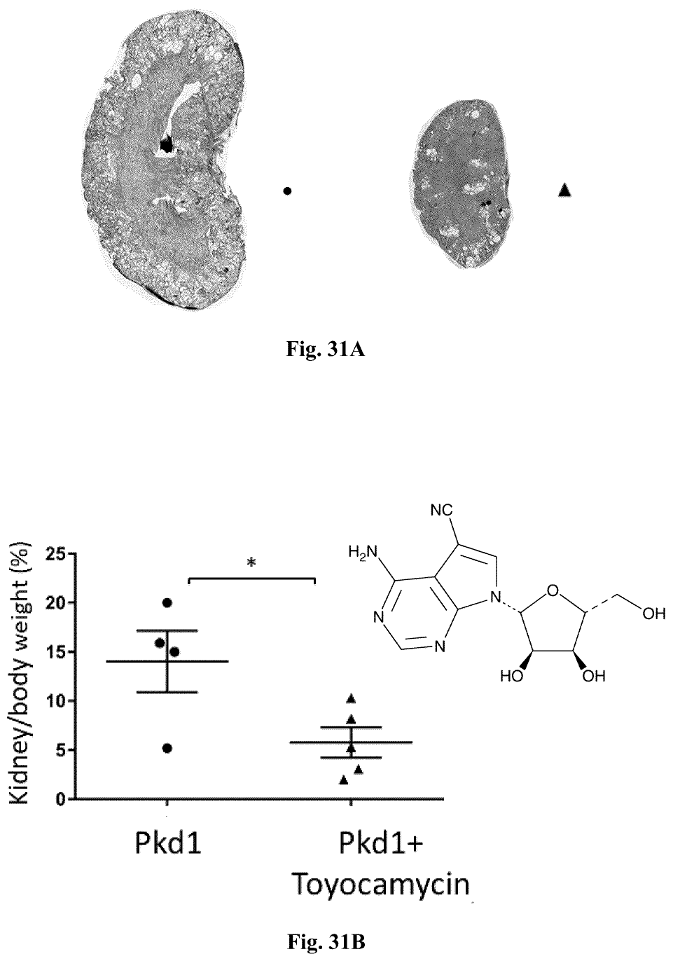

Based on these genetic data, the present study set forth to investigate whether chemical inhibition of Irela endoribonuclease can modulate disease progression in vivo. Toyocamycin (FIGS. 31A-31B) is a natural adenosine analog originally isolated from Streptomyces toyocaensis. It was shown to inhibit Ire1α endoribonuclease activity with an IC₅₀ of ˜100 nM in vitro without any impact on nucleotide incorporation up to concentrations of 10 uM. Furthermore, toyocamycin was previously tested in a Phase 1 trial with no apparent systemic side effects (no changes in Hb, WBC, Pt, AP, SGOT and BUN).

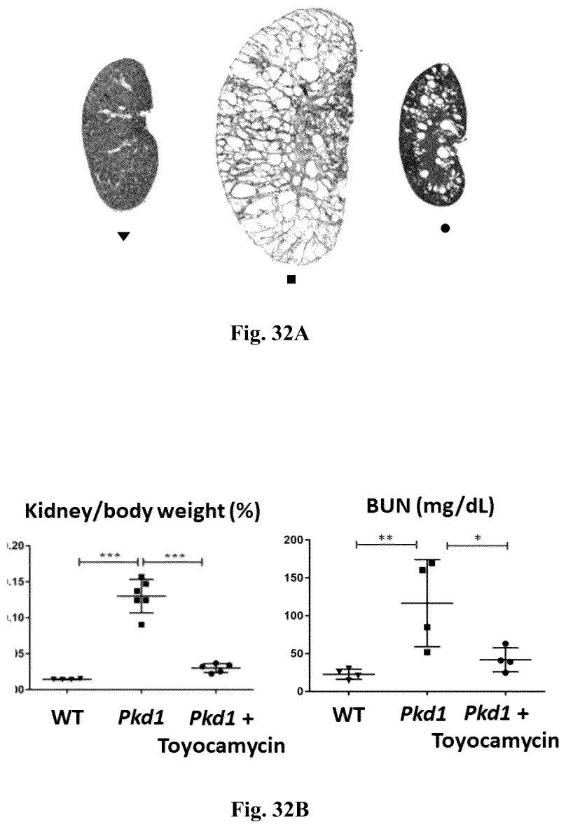

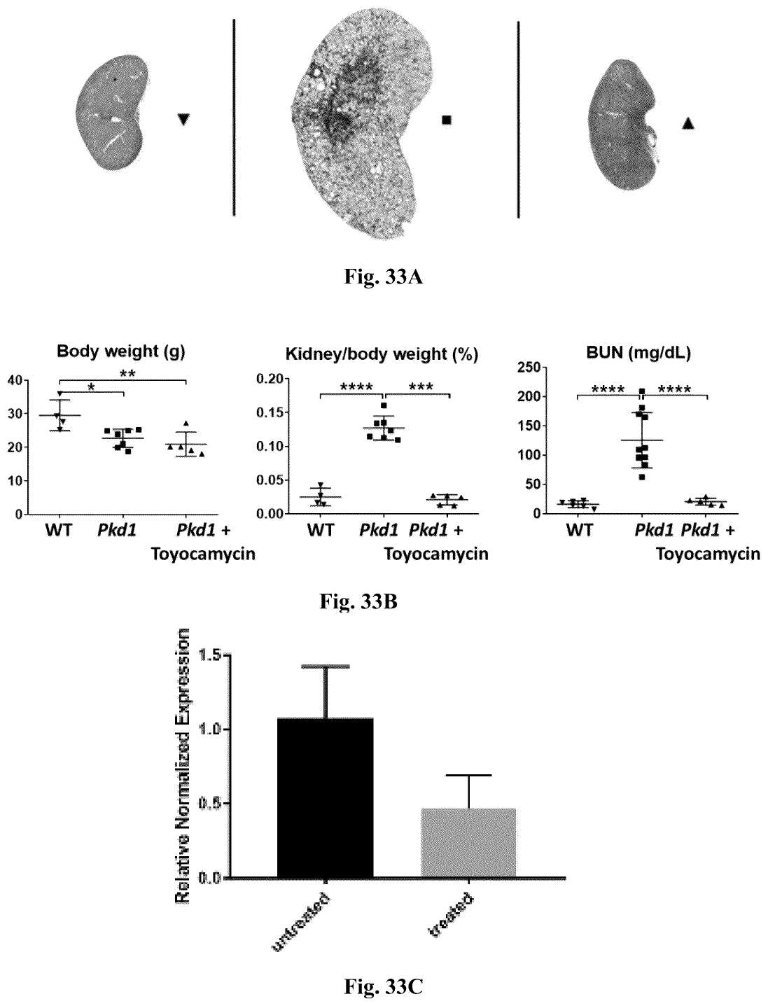

The present study first tested toyocamycin in Pkd1^{flox/flox}; Pax8rtTA; tet-OCre mice. Animals were given doxycycline water between P28 to P42 then treatment was started from week 6 until week 18 (0.5 mg/kg; once every 2 weeks; 6 i.p. injections). As seen in FIGS. 32A-32B, toyocamycin treatment resulted in a significant decrease in KW/BW ratio (*p=0.03) compared with the untreated mice. The present study recapitulated these findings in another slow progressing adult ADPKD model. A compound mutant model with a floxed Pkd1 allele in trans with a human pathogenic hR2220W (mR2216W) missense mutation that causes defects in PC1 GPS cleavage and trafficking was used. Pkd1^{R2216W/flox} line was crossed with either Pkhd1-Cre or Pax8rtTA; Tet-OCre to generate neonatal or adult doxycycline inducible lines respectively. As seen in FIGS. 33A-33B, treatment of Pkd1R2216W/flox;Pkhd1-Cre mice with toyocamycin at P10 (1 dose; 0.5 mg/kg via i.p.) leads to a dramatic improvement in the phenotype as assessed at P16 via KW/BW ratio (***p<0.001) and BUN (*p<0.05). Similarly, treatment of adult Pkd1^{R2216W/flox}; Pax8rtTA; tet-OCre mice (induced between P28 and P42 with doxycycline water) with toyocamycin once every 2 weeks for 12 weeks (between week 6 and 18; 6 doses via i.p.) results in almost a complete rescue in disease progression as assessed via KW/BW ratio (***p<0.001) and BUN (***p<0.001). Gratifyingly, body weight did not differ between the untreated vs. treated animals suggesting that the drug is well tolerated (FIGS. 33A-33B).

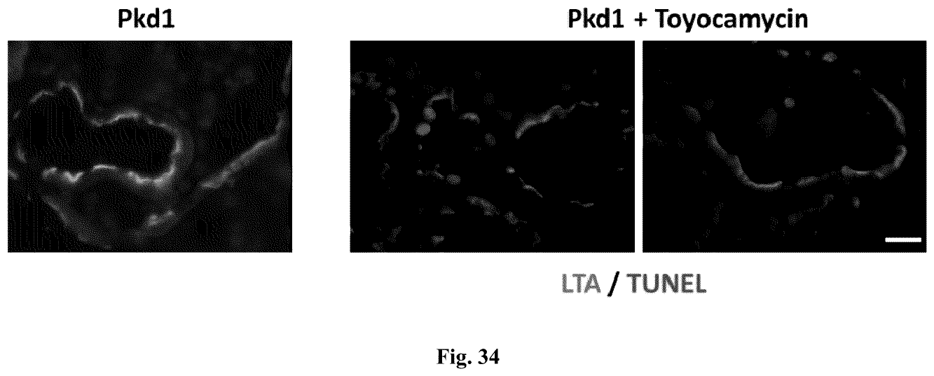

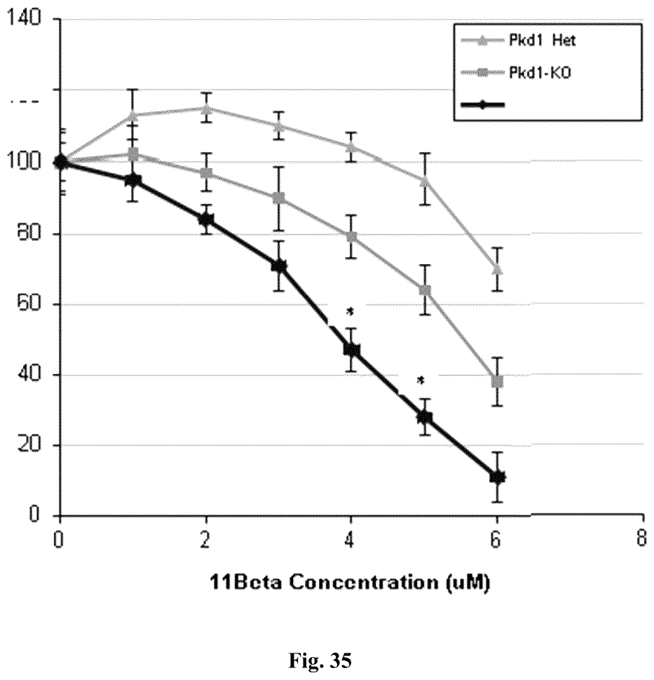

When examined the levels of spliced XBP1, they were lower in the toyocamycin treated vs. untreated mice suggesting target-engagement i.e. inhibition of Ire1α endoribonuclease activity (upper right inset; FIG. 33A). Finally, the present study examined the status of apoptosis in kidneys isolated from Pkd1^{R2216W/flox}; Pax8rtTA; tet-OCre mice treated with toyocamycin between week 12 to 18 (FIG. 34). While untreated mice do not exhibit any apoptosis, toyocamycin treated kidneys show a specific TUNEL-positive signal in cyst lining cells (from proximal tubules marked by LTA). Preliminary analysis of cells where Cre is not expressed suggests that the effect of toyocamycin is restricted to Pkd1 null segments (data not shown). Finally, the present study set forth to examine whether the effect seen in mice can be recapitulated with a relevant cell line model. Although cell line models for PKD are average mimics of the animal models, they do provide opportunities for a closer look at mechanisms of genotype-phenotype relationships in the context of programmed cell death. The present study used a previously described cell line i.e. PN24; Pkd1^{-/-}) as the starting point for inactivation of XBP1 via CRISPR/Cas9. At baseline, no differences in cell viability among the 3 genotypes i.e. Pkd1^{het}, Pkd1^{-/-}, and Pkd1/XBP1 DKO were observed. The present study then compared the response of Pkd1^{+/-}, Pkd1^{-/-}, and Pkd1/XBP1 DKO cells to a sensitized background induced by treatment with 11β-dichloro, an ROS inducer. As seen in FIG. 35, dose response viability curves measured by a metabolic fluorescent dye (Cell-titer Blue) reveal that the Pkd1/XBP1 DKO cells are particularly sensitive to apoptosis on this background as compared with either Pkd1 SKO or heterozygous cells. These data suggest that using appropriate genetic cell models on a sensitized background may be useful for dissecting the various pathways that render the Pkd1/XBP1 DKO cells prone to apoptosis.

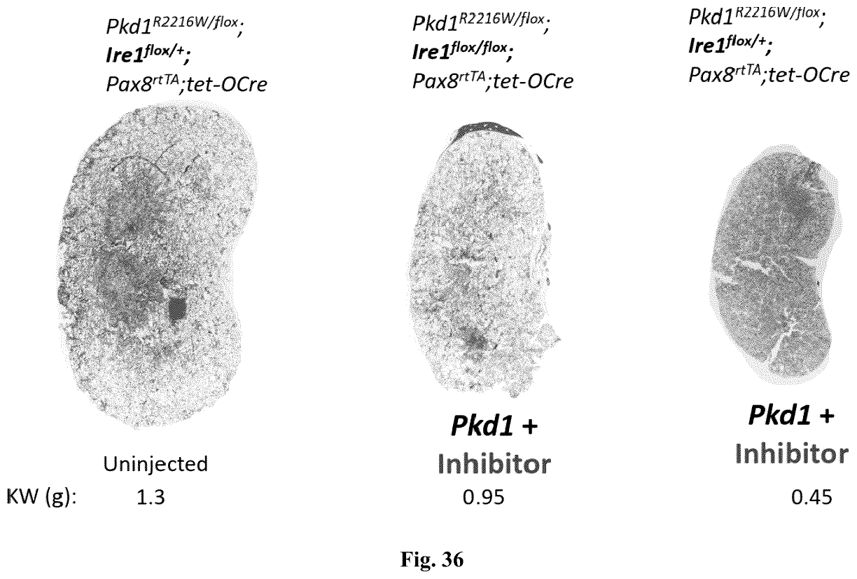

The effect of toyocamycin depends on the presence of Ire1, as shown in FIG. 36.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

### ENUMERATED EMBODIMENTS

The following enumerated embodiments are provided, the numbering of which is not to be construed as designating levels of importance:

Embodiment 1: A method of treating or preventing Autosomal Dominant Polycystic Kidney Disease (ADPKD) and/or Autosomal Dominant Polycystic Liver Disease (ADPLD) and/or Autosomal Recessive Polycystic Kidney Disease (ARPKD) in a mammal, the method comprising administering to the mammal a therapeutically effective amount of an IRE1α endonuclease activity inhibitor.

Embodiment 2: The method of embodiment 1, wherein the inhibitor does not inhibit IRE1α kinase activity.

Embodiment 3: The method of any one of embodiments 1-2, wherein the inhibitor comprises a peptide, antibody, small molecule, antagonist, aptamer, or peptidomimetic, and any combinations thereof.

Embodiment 4: The method of any one of embodiments 1-3, wherein the small molecule comprises toyocamycin, or a salt, solvate, tautomer, geometric isomer, enantiomer, and/or diastereoisomer thereof.

Embodiment 5: The method of any one of embodiments 1-4, wherein the administration has at least one of the following effects; (a) slows or inhibits development of at least one cyst in the mammal's kidney or liver; (b) kills or prevent growth or multiplication of at least one cyst cell; (c) selectively kills or prevents growth or multiplication of at least one cyst cell as compared to at least one non-cyst cell.

Embodiment 6: The method of any one of embodiments 1-5, wherein the inhibitor is the only therapeutically effective agent administered to the mammal.

Embodiment 7: The method of any one of embodiments 1-6, wherein the inhibitor is the only therapeutically effective agent administered to the mammal in an amount sufficient to treat or prevent ADPKD and/or ADPLD and/or ARPKD in the mammal.

Embodiment 8: The method of any one of embodiments 1-7, wherein the mammal is further administered at least one additional agent that treats or prevents ADPKD and/or ADPLD and/or ARPKD.

Embodiment 9: The method of embodiment 8, wherein the at least one additional agent is Tolvaptan, or a salt, solvate, tautomer, geometric isomer, enantiomer, and/or diastereoisomer thereof.

Embodiment 10: The method of any one of embodiments 1-9, wherein the mammal is human.

Embodiment 11: The method of any one of embodiments 1-10, wherein the inhibitor is administered to the mammal by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous routes.

What is claimed is:

1. A method of treating or preventing Autosomal Dominant Polycystic Kidney Disease (ADPKD) and/or Autosomal Dominant Polycystic Liver Disease (ADPLD) and/or Autosomal Recessive Polycystic Kidney Disease (ARPKD) in a mammal, the method comprising administering to the mammal a therapeutically effective amount of an IRE1α endonuclease activity inhibitor.

2. The method of claim 1, wherein the inhibitor does not inhibit IRE1α kinase activity.

3. The method of claim 1, wherein the inhibitor comprises a peptide, antibody, small molecule, antagonist, aptamer, or peptidomimetic, and any combinations thereof.

4. The method of claim 3, wherein the small molecule comprises toyocamycin, or a salt, solvate, tautomer, geometric isomer, enantiomer, and/or diastereoisomer thereof. 5 The method of claim 1, wherein the administration has at least one of the following effects; (a) slows or inhibits development of at least one cyst in the mammal's kidney or liver; (b) kills or prevent growth or multiplication of at least one cyst cell; (c) selectively kills or prevents growth or multiplication of at least one cyst cell as compared to at least one non-cyst cell.

6. The method of claim 1, wherein the inhibitor is the only therapeutically effective agent administered to the mammal.

7. The method of claim 1, wherein the inhibitor is the only therapeutically effective agent administered to the mammal in an amount sufficient to treat or prevent ADPKD and/or ADPLD and/or ARPKD in the mammal.

8. The method of claim 1, wherein the mammal is further administered at least one additional agent that treats or prevents ADPKD and/or ADPLD and/or ARPKD.

9. The method of claim 8, wherein the at least one additional agent is Tolvaptan, or a salt, solvate, tautomer, geometric isomer, enantiomer, and/or diastereoisomer thereof.

10. The method of claim 1, wherein the mammal is human.

11. The method of claim 1, wherein the inhibitor is administered to the mammal by at least one route selected from the group consisting of nasal, inhalational, topical, oral, buccal, rectal, pleural, peritoneal, vaginal, intramuscular, subcutaneous, transdermal, epidural, intratracheal, otic, intraocular, intrathecal, and intravenous routes.
